(19) United States
(12) Patent Application Publication
(10) Pub. No.: US 2011/0286998 A1
(43) Pub. Date: Nov. 24, 2011

(54) METHODS FOR TREATING AND PREVENTING CANCERS THAT EXPRESS THE HYPOTHALAMIC-PITUITARY-GONADAL AXIS OF HORMONES AND RECEPTORS

(75) Inventors: Christopher W. Gregory, Cary, NC (US); Maria E. Kononov, Raleigh, NC (US); Eric S. Werdin, Morrisville, NC (US); James Lester Barbee, IV, Hillsborough, NC (US); Carol Giamario, Cary, NC (US)

(73) Assignee: Voyager Pharmaceutical Corporation, Swampscott, MA (US)

(21) Appl. No.: 12/950,913

(22) Filed: Nov. 19, 2010

Related U.S. Application Data
(63) Continuation of application No. 12/007,576, filed on Jan. 11, 2008, now abandoned.
Application No. 12/007,576 is a continuation of application No. 11/385,668, filed on Mar. 22, 2006, now abandoned.
Application No. 11/385,668 is a continuation-in-part of application No. 11/180,667, filed on Jul. 14, 2005, now abandoned.
Application No. 11/180,667 is a continuation-in-part of application No. 11/180,668, filed on Jul. 14, 2005, now abandoned.
Application No. 11/180,668 is a continuation-in-part of application No. 10/321,579, filed on Dec. 18, 2002, now abandoned.
(60) Provisional application No. 60/340,502, filed on Dec. 19, 2001.
(60) Provisional application No. 60/369,857, filed on Apr. 5, 2002.
(60) Provisional application No. 60/383,624, filed on May 29, 2002.
(60) Provisional application No. 60/385,577, filed on Jun. 5, 2002.
(60) Provisional application No. 60/385,560, filed on Jun. 5, 2002.
(60) Provisional application No. 60/385,559, filed on Jun. 5, 2002.
(60) Provisional application No. 60/385,561, filed on Jun. 5, 2002.
(60) Provisional application No. 60/385,575, filed on Jun. 5, 2002.
(60) Provisional application No. 60/385,576, filed on Jun. 5, 2002.

(51) Int. Cl. A61K 39/395; A61K 31/56; A61K 38/18; A61P 35/00; A61K 31/715; A61K 31/568; A61K 39/00; A61K 38/24; A61K 38/17

(52) U.S. Cl. 424/130.1; 514/10.1; 514/10.3; 514/171; 514/169; 514/7.6; 514/20.7; 514/54; 514/178; 424/184.1

(57) ABSTRACT

Methods are provided for treating HPG axis-positive cancers, preventing or slowing proliferation of cells of HPG axis-positive cancer origin, preventing HPG axis-positive cancers in a patient at risk of contracting such cancers, preventing or inhibiting an upregulation of the cell cycle in HPG axis-positive cancer-derived cells in a patient, and decreasing the level of HPG axis-positive cancer-specific markers in a patient.

### RELATED APPLICATIONS

This application is a continuation of U.S. patent application Ser. No. 12/007,576, which is a continuation of U.S. patent application Ser. No. 11/385,668, filed Mar. 22, 2006, entitled Methods for Treating and Preventing Cancers That Express The Hypothalamic-Pituitary-Gonadal Axis of Hormones and Receptors.

U.S. patent application Ser. No. 12/007,576 is a continuation of U.S. patent application Ser. No. 11/385,668, filed Mar. 22, 2006. U.S. patent application Ser. No. 11/385,668 is a continuation-in-part of U.S. patent application Ser. Nos. 11/180,667 and 11/180,668, filed Jul. 14, 2005. U.S. patent application Ser. No. 11/180,668 is also a continuation-in-part of U.S. patent application Ser. No. 10/321,579, filed Dec. 18, 2002, which claims priority to U.S. Provisional Application No. 60/340,502, filed Dec. 19, 2001; U.S. Provisional Application No. 60/369,857, filed Apr. 5, 2002; U.S. Provisional Application No. 60/383,624, filed May 29, 2002; U.S. Provisional Application No. 60/385,577, filed Jun. 5, 2002; U.S. Provisional Application No. 60/385,576, filed Jun. 5, 2002; U.S. Provisional Application No. 60/385,560, filed Jun. 5, 2002; U.S. Provisional Application No. 60/385,559, filed Jun. 5, 2002; U.S. Provisional Application No. 60/385,561, filed Jun. 5, 2002; and U.S. Provisional Application No. 60/385,575, filed Jun. 5, 2002.

The entirety of each of the above-identified applications is hereby incorporated by reference herein.

### FIELD OF THE INVENTION

The present invention relates to methods for treating, preventing, delaying, or mitigating HPG axis-positive cancers, for decreasing the level of HPG axis-positive cancer-specific markers, and for preventing or slowing proliferation of malignant cells of HPG axis-positive cancers.

### BACKGROUND

Gonadotropin releasing hormone (GnRH) receptor-positive cancers are derived at many different sites in the body. Cancers that express GnRH receptors include the following: prostate, brain (including but not limited to glioblastoma, astrocytoma, medulloblastoma, neuroblastoma, meningioma), breast, ovary, endometrial, pancreas, lung, malignant melanoma, renal cell carcinoma, hepatocarcinoma, oral carcinoma, laryngeal carcinoma, angiomyxoma, and colon cancer.

Normal as well as cancerous prostate tissue expresses hormones involved in the hypothalamic-pituitary-gonadal (HPG) axis and their respective cognate receptors. These hormones include activins, inhibins, follistatin, GnRH, follicle stimulating hormone (FSH), luteinizing hormone (LH), and sex steroids. It was estimated for the year 2005 that a total of 232,090 new prostate cancers would be diagnosed and 30,350 deaths would be attributed to prostate cancers in the United States (American Cancer Society, Cancer Facts and Figures. 2005. Atlanta: American Cancer Society; 2005).

Receptors for luteinizing hormone releasing hormone (LHRH) have been detected in meningiomata, glioblastoma multiforme, gliomata, and chordoma using LHRH binding assays, demonstrating a possible autocrine signaling loop in brain cancers (van Groeninghen J C, Kiesel L, Winkler D, Zwirner M. Effects of luteinising-hormone-releasing hormone on nervous-system tumors. Lancet 352:372-373, 1998). It was estimated for the year 2005 that a total of 18,500 new brain cancers would be diagnosed and 12,760 deaths would be attributed to brain cancers in the United States (American Cancer Society, Cancer Facts and Figures. 2005. Atlanta: American Cancer Society; 2005).

Immunoreactivity for GnRH receptors has been detected in the cytoplasm of breast carcinoma cells from invasive ductal carcinoma and positively correlated with estrogen and progesterone receptor labeling indices (Moriya T, Suzuki T, Pilichowska M, Ariga N, Kimura N, Ouchi N, Nagura H, Sasano H. Immunohistochemical expression of gonadotropin releasing hormone receptor in human breast carcinoma. Pathol. Int. 51:333-337, 2001). The expression of GnRH and GnRH receptors has been demonstrated in breast cancer at the protein and gene level (reviewed in Emons G, Grundker C, Gunthert A R, Westphalen S, Kavanagh J, Verschraegen C. GnRH antagonists in the treatment of gynecological and breast cancers. Endocrine-Related Cancer 10:291-299, 2003). It was estimated for the year 2005 that a total of 212,930 new breast cancers would be diagnosed and 40,870 deaths would be attributed to breast cancers in the United States (American Cancer Society, Cancer Facts and Figures. 2005. Atlanta: American Cancer Society; 2005).

As many as 70% of ovarian cancers have been shown to express GnRH and its receptor at the protein and gene levels (Grundker C, Gunthert A R, Westphalen S, Emons G. Biology of the gonadotropin-releasing hormone system in gynecological cancers. Eur. J. Endocrinol. 146:1-14, 2002). It was estimated for the year 2005 that a total of 22,220 new ovarian cancers would be diagnosed and 16,210 deaths would be attributed to ovarian cancers in the United States (American Cancer Society, Cancer Facts and Figures. 2005. Atlanta: American Cancer Society; 2005).

GnRH and its receptor have been shown to be expressed in as many as 80% of endometrial cancers at the protein and gene levels (Volker P, Grundker C, Schmidt O, Schulz K D, Emons G. Expression of receptors for luteinizing hormone-releasing hormone in human ovarian and endometrial cancers: frequency, autoregulation, and correlation with direct antiproliferative activity of luteinizing hormone-releasing hormone analogs. Am. J. Obstetr. Gynecol. 186:171-179, 2002). It was estimated for the year 2005 that a total of 40,880 new uterine cancers would be diagnosed and 7,310 deaths would be attributed to uterine cancers in the United States (American Cancer Society, Cancer Facts and Figures. 2005. Atlanta: American Cancer Society; 2005).

Pancreatic cancer induced by N-nitrosobis(2-oxopropyl)amine in hamsters also has been shown to express GnRH receptors (Fekete M, Zalatnai A, Schally A V. Presence of membrane binding sites for [D-TRP6]-luteinizing hormone-releasing hormone in experimental pancreatic cancer. Cancer Lett. 45:87-91, 1989). Additional studies demonstrated the presence of GnRH receptors in 67% of patients with chronic pancreatitis and in 57% of patients with pancreatic cancer (Friess H, Buchler M, Kiesel L, Kruger M, Beger H G. LH-RH receptors in the human pancreas. Basis for antihormonal treatment in ductal carcinoma of the pancreas. Int. J. Pancreatol. 10:151-159, 1991). It was estimated for the year 2005 that a total of 32,180 new pancreatic cancers would be diagnosed and 31,800 deaths would be attributed to pancreatic cancers in the United States (American Cancer Society, Cancer Facts and Figures. 2005. Atlanta: American Cancer Society; 2005).

GnRH receptor expression was demonstrated by reverse transcriptase-polymerase chain reaction in normal lung tissue (Tieva A, Stattin P, Wikstrom P, Bergh A, Damber J E. Gonadotropin-releasing hormone receptor expression in the human prostate. Prostate 47:276-284, 2001). A published study using a Pseudomonas exotoxin-based chimeric toxin aimed at targeting cancer cells bearing GnRH receptors demonstrated that primary cultures of lung adenocarcinoma were growth-inhibited and even killed by the conjugated toxin. This work provided evidence of GnRH receptor expression in lung cancer (Nechushtan A, Yarkoni S, Marianovsky I, Lorberboum-Galski H. Adenocarcinoma cells are targeted by the new GnRH-PE66 chimeric toxin through specific gonadotropin-releasing hormone binding sites. J. Biol. Chem. 272:11597-11603, 1997). It was estimated for the year 2005 that a total of 172,570 new lung cancers would be diagnosed and 163,510 deaths would be attributed to lung cancers in the United States (American Cancer Society, Cancer Facts and Figures. 2005. Atlanta: American Cancer Society; 2005).

GnRH receptors were shown to be expressed by human malignant melanoma cell lines at the gene and protein levels (Moretti R M, Montagnani Marelli M, Van Groeninghen J C, Limonta P. Locally expressed LHRH receptors mediate the oncostatic and antimetastatic activity of LHRH agonists on melanoma cells. J. Clin. Endocrinol. Metab. 87:3791-3797, 2002). Further, GnRH receptor expression was demonstrated in 19 of 19 human melanoma tissue specimens derived from primary tumors and metastases (Keller G, Schally A V, Gaiser T, Nagy A, Baker B, Westphal G, Halmos G, Engel J B. Human malignant melanomas express receptors for luteinizing hormone releasing hormone allowing targeted therapy with cytotoxic luteinizing hormone releasing hormone analogue. Cancer Res. 65:5857-5863). It was estimated for the year 2005 that a total of 59,580 new melanoma cancers would be diagnosed and 7,770 deaths would be attributed to melanoma cancers in the United States (American Cancer Society, Cancer Facts and Figures. 2005. Atlanta: American Cancer Society; 2005).

A published study using a Pseudomonas exotoxin-based chimeric toxin aimed at targeting cancer cells bearing GnRH receptors demonstrated that primary cultures of renal cell adenocarcinoma were growth-inhibited and even killed by the conjugated toxin. This work provided evidence of GnRH receptor expression in renal cell cancer (Nechushtan A, Yarkoni S, Marianovsky I, Lorberboum-Galski H. Adenocarcinoma cells are targeted by the new GnRH-PE66 chimeric toxin through specific gonadotropin-releasing hormone binding sites. J. Biol. Chem. 272:11597-11603, 1997). Further, GnRH receptor expression was demonstrated in 37 of 37 human renal cell carcinomas derived from primary tumors and metastases (Keller G, Schally A V, Gaiser T, Nagy A, Baker B, Halmos G, Engel J B. Receptors for luteinizing hormone releasing hormone expressed on human renal cell carcinomas can be used for targeted chemotherapy with cytotoxic luteinizing hormone releasing hormone analogues. Clin. Cancer Res. 11:5549-5557, 2005). It was estimated for the year 2005 that a total of 36,160 new renal cancers would be diagnosed and 12,660 deaths would be attributed to renal cancers in the United States (American Cancer Society, Cancer Facts and Figures. 2005. Atlanta: American Cancer Society; 2005).

GnRH receptor expression and hormone binding was demonstrated in normal human liver and in a human hepatocarcinoma cell line (Pati D, Habibi H R. Inhibition of human hepatocarcinoma cell proliferation by mammalian and fish gonadotropin-releasing hormones. Endocrinol. 136:75-84, 1995). A published study using a Pseudomonas exotoxin-based chimeric toxin aimed at targeting cancer cells bearing GnRH receptors demonstrated that a liver cancer cell line was growth-inhibited and even killed by the conjugated toxin. This work provided evidence of GnRH receptor expression in liver cancer (Nechushtan A, Yarkoni S, Marianovsky I, Lorberboum-Galski H.

Adenocarcinoma cells are targeted by the new GnRH-PE66 chimeric toxin through specific gonadotropin-releasing hormone binding sites. J. Biol. Chem. 272:11597-11603, 1997). It was estimated for the year 2005 that a total of 17,550 new liver cancers would be diagnosed and 15,420 deaths would be attributed to liver cancers in the United States (American Cancer Society, Cancer Facts and Figures. 2005. Atlanta: American Cancer Society; 2005).

In the hamster cheek pouch carcinoma model of oral cancer, GnRH receptors appear during progression of the cancer (Crean D H, Liebow C, Lee M T, Kamer A R, Schally A V, Mang T S. Alterations in receptor-mediated kinases and phosphatases during carcinogenesis. J. Cancer Res. Clin. Oncol. 121:141-149, 1995). GnRH receptor binding was demonstrated in oral carcinoma and laryngeal carcinoma cell lines (Kreb L J, Wang X, Nagy A, Schally A V, Prasad P N, Liebow C. A conjugate of doxorubicin and an analog of luteinizing hormone-releasing hormone shows increased efficacy against oral and laryngeal cancers. Oral Oncol. 38:657-663, 2002). It was estimated for the year 2005 that a total of 29,370 new oral cancers would be diagnosed and 7,320 deaths would be attributed to oral cancers in the United States (American Cancer Society, Cancer Facts and Figures. 2005. Atlanta: American Cancer Society; 2005).

Recurrent aggressive angiomyxomas of the perineum or vulva, while rare, have been treated with GnRH agonists with some success, indicating that GnRH receptors are expressed and bind GnRH agonists (Shinohara N, Nonomura K, Ishikawa S, Seki H, Koyanagi T. Medical management of recurrent aggressive angiomyxoma with gonadotropin-releasing hormone agonist. Int. J. Urol. 11:432-435, 2004). Estimated new cases in the United States for 2005 were 3,870 and 2,140, respectively, for vulvar and vaginal cancers (American Cancer Society, Cancer Facts and Figures. 2005. Atlanta: American Cancer Society; 2005).

GnRH receptor expression was detected by reverse transcriptase-polymerase chain reaction in normal colon tissue (Tieva A, Stattin P, Wikstrom P, Bergh A, Damber J E. Gonadotropin-releasing hormone receptor expression in the human prostate. Prostate 47:276-284, 2001). A published study using a Pseudomonas exotoxin-based chimeric toxin aimed at targeting cancer cells bearing GnRH receptors demonstrated that colon cancer cell lines and primary cultures from colon cancers were growth-inhibited and even killed by the conjugated toxin. This work provided evidence of GnRH receptor expression in colon cancer (Nechushtan A, Yarkoni S, Marianovsky I, Lorberboum-Galski H. Adenocarcinoma cells are targeted by the new GnRH-PE66 chimeric toxin through specific gonadotropin-releasing hormone binding sites. J. Biol. Chem. 272:11597-11603, 1997). It was estimated for the year 2005 that a total of 104,950 new colon cancers would be diagnosed and 56,290 deaths would be attributed to colon cancers in the United States (American Cancer Society, Cancer Facts and Figures. 2005. Atlanta: American Cancer Society; 2005).

There is a need in the art for therapeutically effective treatments and preventative measures for HPG axis-positive cancers such as prostate cancer, brain cancer (including but not limited to glioblastoma, astrocytoma, medulloblastoma, neuroblastoma, and meningioma), breast cancer, ovarian cancer, endometrial cancer, pancreatic cancer, lung cancer, malignant melanoma, renal cell carcinoma, hepatocarcinoma, oral carcinoma, laryngeal carcinoma, angiomyxoma, and colon cancer. The present invention provides such treatments and measures.

### DEFINITIONS

As used in this specification, the term “autocrine” refers to secretion of a factor that stimulates the secretory cell itself.

“Endocrine” refers to secretion (as of an endocrine gland) that is transmitted by blood to a tissue on which the secretion has its specific effect.

“Paracrine” refers to a form of signaling in which the target cell is physically close to the signal-releasing cell.

“Chemical castration” refers to use of a GnRH analogue to reduce serum levels of testosterone to “castrate levels,” which is typically considered to be less than or equal to about 50 ng/dL of testosterone.

“HPG axis” refers to the hypothalamic-pituitary-gonadal endocrine feedback loop through which the production of sex steroids (estrogen and testosterone) is regulated. GnRH is produced by hypothalamic cells and binds to gonadotrope cells in the pituitary which produce the gonadotropins (LH and FSH) which then bind to cognate receptors in the ovaries and testes to cause production of estrogen and testosterone, respectively.

As used with reference to the HPG axis, the term “therapeutically effective” means that an amount of an agent or a combination of agents is effective to reduce or suppress local tissue production of hormones of the HPG axis (i.e., effective to cause a paracrine or autocrine effect on the target tissue).

“Physiologically equivalent dose” refers to a dose of a second physiological agent that achieves the same or similar physiological responses as a dose of a first physiological agent.

### SUMMARY

While GnRH receptors have been demonstrated in various cancers, data presented herein demonstrates that GnRH, LH, LH receptor, FSH, and FSH receptor are also expressed in multiple cancers, thus indicating an autocrine/paracrine signaling mechanism that could be blocked by using sufficiently high doses of GnRH analogues to achieve elevated tissue levels of the analogues.

The present invention provides that suppression of autocrine/paracrine signaling in HPG axis-positive cancers requires doses of GnRH agonists that are significantly higher than those required to suppress endocrine GnRH signaling at the level of the pituitary. The present invention further provides that hormones of the hypothalamic-pituitary-gonadal (HPG) axis function not only in an endocrine fashion to modulate cancer cell function but also in an autocrine/paracrine fashion to regulate cancer cell function. While customary doses of GnRH agonists and antagonists may generally be considered to be adequate to suppress endocrine influences of hormones of the HPG axis by lowering their serum concentrations, these same doses of GnRH antagonists and agonists are believed to be subtherapeutic when it comes to adequately suppressing local tissue production of these hormones. In this specification, the term “therapeutically effective” as used with reference to the HPG axis means that an amount of an agent or a combination of agents is effective to reduce or suppress local tissue production of hormones of the HPG axis. For example, a therapeutically effective amount of a GnRH agonist as used in the present invention for treatment of prostate cancer is expected to be higher than the current doses used in the treatment, prevention, mitigation, or slowing of the progress of prostate cancer.

Examples of GnRH analogues that are useful in the present invention include leuprolide, triptorelin, buserelin, nafarelin, desorelin, histrelin, and goserelin. Other LH/FSH-inhibiting agents that can be used according to the invention include GnRH antagonists, GnRH receptor blockers, such as cetrorelix and abarelix, and LH or FSH receptor blockers. Currently approved GnRH agonists and antagonists, dosage levels, and plasma/serum levels of active medication (according to package inserts and prescribing information) are as follows: LUPRON® DEPOT 3.75 mg 1 month injection gives a mean plasma leuprolide concentration of 4.6-10.2 ng/ml at 4 hours postdosing; LUPRON® DEPOT 7.5 mg 1 month injection gives a mean plasma leuprolide concentration of 20 ng/ml at 4 hours and 0.36 ng/ml at 4 weeks; LUPRON® DEPOT-PED 11.25 mg 1 month injection gives a mean plasma leuprolide concentration of 1.25 ng/ml at 4 weeks; LUPRON® DEPOT-PED 15 mg injection gives a mean plasma leuprolide concentration of 1.59 ng/ml at 4 weeks; LUPRON® DEPOT 22.5 mg 3 month injection gives a mean plasma leuprolide concentration of 48.9 ng/ml at 4 hours and 0.67 ng/ml at 12 weeks; LUPRON® DEPOT 30 mg 4 month injection gives a mean plasma leuprolide concentration of 59.3 ng/ml at 4 hours and 0.3 ng/ml at 16 weeks; VIADUR® 72 mg 12 month implantation gives a mean serum leuprolide concentration of 16.9 ng/ml at 4 hours and 2.4 ng/ml at 24 hours with a 0.9 ng/ml mean serum concentration for 12 months; ELIGARD® 7.5 mg 1 month injection gives a mean serum leuprolide concentration of 25.3 ng/ml at 5 hours and a serum level range of 0.28-2.0 ng/ml for one month; ZOLADEX® 3.6 mg 1 month gives a mean serum concentration of 3 ng/ml at 15 days and 0.5 ng/ml at 30 days; ZOLADEX® 10.8 mg 3 month gives a mean serum concentration of 8 ng/ml on the first day after dosing and thereafter, mean concentrations remain relatively stable in the range of 0.3 to 1 ng/ml to the end of the dosing period; SYNAREL® 200 micrograms gives a peak serum nafarelin concentration range of 0.2-1.4 ng/ml, whereas a single dose of 800 micrograms gives a peak serum concentration range of 0.5 to 5.3 ng/ml; TRELSTAR DEPOT 3.75 mg 1 month gives a mean plasma triptorelin concentration of 28.43 ng/ml at 4 hours and declines to 0.084 ng/ml at 4 weeks; Supprelin 200 μg/ml, 500 μg/ml and 1000 μg/ml for daily injection; SUPREFACT® 6.3 mg 2 month implant or 500 μg every 8 hours for 7 days followed by 200 μg per day; CETROTIDE® 0.25 mg daily or 3.0 mg every 4 days gives a mean plasma cetrorelix concentration of 4.97 ng/ml or 28.5 ng/ml at 4 hours, respectively; PLENAXIS® 100 mg given on days 1, 15, and 28 and every 4 weeks afterward gives a peak concentration of abarelix of 43.4 ng/ml 3 days after dosing and maintains 94% of men studied at castrate levels of androgen (≦50 ng/dL) during the dosing period; ANTAGON 250 μg daily gives a mean plasma ganirelix concentration of 14.8 ng/ml at 4 hours. The GnRH analogues plasma levels listed above are generally considered sufficient in prostate cancer patients to achieve the desired endocrine effects of reducing serum androgens to below castrate levels (≦50 ng/dL), resulting in chemical castration. The present invention makes use of therapeutically effective amounts of agents or combinations of agents to reduce or suppress local tissue production of hormones of the HPG axis (i.e., effective to cause a paracrine or autocrine effect on the target tissue).

GnRH agonists were developed as a method of suppressing sex steroid production as an alternative to surgical castration in the treatment of advanced prostate cancer. GnRH agonists are analogues of the endogenous GnRH decapeptide with specific amino acid substitutions. Replacement of the GnRH carboxyl-terminal glycinamide residue with an ethylamide group greatly increases the affinity of these analogues for the GnRH receptor compared to the endogenous peptide. Many of these analogues also have a longer half-life than endogenous GnRH. Administration results in an initial increase in serum gonadotropin concentrations that persists for several days (there is also a corresponding increase in testosterone in men and in estrogen in pre-menopausal women). This is followed by a precipitous decrease in gonadotropins and sex steroids. This suppression is thought to be secondary to the loss of GnRH signaling due to down-regulation of pituitary GnRH receptors (Belchetz, P. E., Plant, T. M., Nakai, Y., Keogh, E. J., and Knobil, E. (1978) Hypophysial responses to continuous and intermittent delivery of hypothalamic gonadotropin-releasing hormone. Science 202:631-633). This is a likely consequence of the increased concentration of ligand, the increased affinity of the ligand for the GnRH receptor, and the continuous receptor exposure to ligand, as opposed to the intermittent exposure that occurs with physiological pulsatile secretion. By this mechanism, chronic administration of GnRH agonists inhibits testicular steroidogenesis, thereby reducing the levels of circulating androgens to castrate levels (≦50 ng/dL). This results in reversible medical castration, a mainstay therapeutic strategy for advanced, metastatic prostate cancer.

GnRH antagonists have also been developed for use in the treatment of prostate cancer. The GnRH antagonists were developed to inhibit gonadotropin and sex steroid synthesis and secretion without the initial spike in gonadotropins and sex steroids associated with GnRH agonists. While GnRH antagonists do prevent this initial burst, there is more “breakthrough” in LH and testosterone secretion than with GnRH agonists (Praecis Pharmaceuticals Incorporated, Plenaxis Package Insert. 2004). This may be due to a compensatory increase in hypothalamic GnRH secretion which alters the ratio of the competing ligands, resulting in activation of the receptor. In contrast, with GnRH agonists, a compensatory increase in hypothalamic GnRH would serve to potentiate receptor down-regulation. In addition to this efficacy issue, GnRH antagonists are associated with occasional anaphylactic reactions due to their high histamine releasing properties (Millar, R. P., Lu, Z. L., Pawson, A. J., Flanagan, C. A., Morgan, K., and Maudsley, S. R. (2004) Gonadotropin-releasing hormone receptors. Endocr. Rev. 25:235-275). Therefore, for chronic use, the GnRH agonists are often preferred as more effective than the GnRH antagonists at suppressing gonadotropins.

As demonstrated in pharmacokinetic studies (FIG. 16), sustained high serum levels of leuprolide acetate (about 5.0-8.0 ng/ml) are achievable using a polymer-based subcutaneous implant formulation. This serum level of drug is considerably higher than the serum levels achieved with currently available depot formulations used to treat advanced prostate cancer. Tumor xenograft studies (FIGS. 17-23) were performed with subcutaneous implants of leuprolide acetate. The high leuprolide serum levels resulted in inhibition or significant slowing of tumor growth compared to placebo control-treated tumors. According to the present invention, high serum levels of leuprolide are expected to result in high local or tissue/tumor levels of leuprolide.

A brief overview of the HPG hormonal axis is presented with reference to FIG. 27. In humans and many other mammals, the centrally produced hormones include gonadotropin releasing hormone (GnRH) from the hypothalamus; and gonadotropins, luteinizing hormone (LH), and follicle stimulating hormone (FSH) from the pituitary. Peripherally produced hormones include estrogen, progesterone, testosterone, and inhibins that are primarily of gonadal origin, while activins and follistatin are produced in all tissues including the gonads (Carr B R, in Williams Textbook of Endocrinology, JD Wilson, DW Foster, HM Kronenberg, and PR Larsen, eds. (Philadelphia Pa., WB Saunders Co.), pp. 751-817 (1998)).

The levels of each of these hormones are regulated by a complex feedback loop. Activins, which are produced by most tissues, stimulate GnRH secretion from the hypothalamus which stimulates the anterior pituitary to secrete the gonadotropins, LH and FSH, which in turn enter the blood stream and bind to receptors in the gonads and stimulate oogenesis/spermatogenesis as well as sex steroid and inhibin production. (Reichlin S. Neuroendocrinology; in Wilson J D, Foster D W, Kronenberg H M, Larsen P R 9eds): William's Textbook of Endocrinology, ed. 9. Philadelphia, Saunders, 1998, pp. 165-248). The sex steroids and inhibin then feedback to the hypothalamus and pituitary, resulting in a decrease in gonadotropin secretion. (Thorner M, Vance M, Laws E Jr., Horvath E, Kovacs K. The anterior pituitary; in Wilson J D, Foster D W, Kronenberg H M, Larsen P R 9eds): William's Textbook of Endocrinology, ed. 9. Philadelphia, Saunders, 1998, pp. 249-340).

Among the goals of the present invention are treatment, mitigation, slowing the progression of, and preventing HPG axis-positive cancers by achieving higher tissue levels of GnRH agonists and/or GnRH antagonists than are currently achieved with available formulations (listed above), whether by administering more of such drugs, by preventing degradation of such drugs once administered, by delivering the drugs at a site where they are needed, by a combination of these methods, or by other methods.

The present invention relates to methods for treating, mitigating, slowing the progression of, or preventing HPG axis-positive cancers, or preventing or slowing proliferation of cells of HPG axis-positive cancer origin, or decreasing the level of a cancer-specific marker in a patient, by administering high doses of at least one physiological agent, such as a GnRH agonist or a GnRH antagonist, that decreases or regulates the blood or tissue levels, expression, production, function, or activity of LH, LH receptors, FSH, FSH receptors, androgenic steroids, androgenic steroid receptors, activins, or activin receptors, or administering a physiological agent that increases or regulates the blood or tissue levels, expression, production, function, or activity of GnRH, GnRH receptors, inhibins, inhibin receptors, beta-glycan, or follistatins.

The invention further encompasses, for example, a method of preventing or inhibiting an upregulation of the cell cycle in HPG axis-positive cancer-derived cells by administering high doses of at least one physiological agent that is a GnRH agonist or antagonist, effective to reduce local tissue production of hormones of the HPG axis or to down-regulate hormone receptors. In embodiments, the physiological agent is leuprolide, and the amount administered is sufficient to maintain serum leuprolide levels at greater than about 1.5 ng/ml for a full dosing period. In other embodiments, the amount of leuprolide administered is sufficient to maintain the serum leuprolide levels at greater than about 2.0 ng/ml for the full dosing period. In further embodiments, the amount of leuprolide administered is sufficient to maintain the serum leuprolide levels at greater than about 2.5 ng/ml for the full dosing period. In still other embodiments, the amount of leuprolide administered is sufficient to maintain the serum leuprolide levels at greater than about 3.0 ng/ml for the full dosing period. In other embodiments, the physiological agent is an agent other than leuprolide, and the amount administered is an amount sufficient to maintain serum levels of the agent at greater than about 1.5 ng/ml for the full dosing period, greater than about 2.0 ng/ml for the full dosing period, or greater than about 2.5 ng/ml for the full dosing period, or greater than about 3.0 ng/ml for the full dosing period. The invention also encompasses, as another example, a method for treating HPG axis-positive cancer in a patient having cancer comprising administering to the patient a physiological agent that decreases the degradation of GnRH agonists or GnRH antagonists, increases the half-life of GnRH agonists or GnRH antagonists, or increases tissue levels of GnRH agonists or GnRH antagonists within the patient.

A “full dosing period” according to the present invention refers to a period of time sufficient to achieve a therapeutic effect in the treatment, mitigation, delay, or prevention of HPG axis-positive cancers, and may be from about one month to about twelve months, or such shorter or longer period of time as is required to achieve the therapeutic effect. In embodiments of the invention, the full dosing period is in the range of from about 30 days to about 90 days. In other embodiments, the full dosing period is about 60 days.

The invention also encompasses, as another example, a method for treating cancer in a patient having HPG axis-positive cancer comprising administering to the patient a physiological agent that decreases the degradation of GnRH agonists or GnRH antagonists, increases the half-life of GnRH agonists or GnRH antagonists, or increases tissue levels of GnRH agonists or GnRH antagonists within the patient.

The present invention additionally encompasses a method for treating HPG axis-positive cancers comprising administering to a patient an initial dose of a GnRH agonist or a GnRH antagonist, monitoring for decreases in an HPG axis-positive cancer-specific marker level in the patient, and subsequently administering to the patient increasing doses of the GnRH agonist or the GnRH antagonist until no further decrease in an HPG axis-positive cancer-specific marker level in the patient is observed.

### BRIEF DESCRIPTION OF THE DRAWINGS

FIG. 1A presents results of an in vitro experiment in which leuprolide acetate was administered to cells of the T47D breast cancer cell line twice daily for a 5-day period.

FIG. 1B presents results of an in vitro experiment in which leuprolide acetate was administered to cells of the T47D breast cancer cell line twice daily for a 5-day period.

FIG. 2A presents results of an in vitro experiment in which leuprolide acetate was administered to cells of the MCF-7 breast cancer cell line twice daily for a 5-day period.

FIG. 2B presents results of an in vitro experiment in which leuprolide acetate was administered to cells of the MCF-7 breast cancer cell line twice daily for a 5-day period.

FIG. 3A presents results of an in vitro experiment in which leuprolide acetate was administered to cells of the H358 lung cancer cell line twice daily for a 5-day period.

FIG. 3B presents results of an in vitro experiment in which leuprolide acetate was administered to cells of the H358 lung cancer cell line twice daily for a 5-day period.

FIG. 3C presents results of an in vitro experiment in which leuprolide acetate was administered to cells of the H358 lung cancer cell line twice daily for a 5-day period.

FIG. 4A presents results of an in vitro experiment in which leuprolide acetate was administered to cells of the H838 lung cancer cell line twice daily for a 5-day period.

FIG. 4B presents results of an in vitro experiment in which leuprolide acetate was administered to cells of the H838 lung cancer cell line twice daily for a 5-day period.

FIG. 4C presents results of an in vitro experiment in which leuprolide acetate was administered to cells of the H838 lung cancer cell line twice daily for a 5-day period.

FIG. 5A presents results of an in vitro experiment in which leuprolide acetate was administered to cells of the HPAC pancreatic cancer cell line twice daily for a 5-day period.

FIG. 5B presents results of an in vitro experiment in which leuprolide acetate was administered to cells of the HPAC pancreatic cancer cell line twice daily for a 5-day period.

FIG. 6 presents average results from two in vitro experiments in which leuprolide acetate was administered to cells of the PANC pancreatic cancer cell line twice daily for a 5-day period.

FIG. 7A presents results of an in vitro experiment in which leuprolide acetate was administered to cells of the CaOV3 ovarian cancer cell line twice daily for a 5-day period.

FIG. 7B presents results of an in vitro experiment in which leuprolide acetate was administered to cells of the CaOV3 ovarian cancer cell line twice daily for a 5-day period.

FIG. 7C presents results of an in vitro experiment in which leuprolide acetate was administered to cells of the CaOV3 ovarian cancer cell line twice daily for a 5-day period.

FIG. 8A presents results of an in vitro experiment in which leuprolide acetate was administered to cells of the SKOV3 ovarian cancer cell line twice daily for a 5-day period.

FIG. 8B presents results of an in vitro experiment in which leuprolide acetate was administered to cells of the SKOV3 ovarian cancer cell line twice daily for a 5-day period.

FIG. 9A presents results of an in vitro experiment in which leuprolide acetate was administered to cells of the MV4-11 leukemia cell line twice daily for a 3-day period.

FIG. 9A presents results of an in vitro experiment in which leuprolide acetate was administered to cells of the MV4-11 leukemia cell line twice daily for a 5-day period.

FIG. 9C presents results of an in vitro experiment in which leuprolide acetate was administered to cells of the MV4-11 leukemia cell line twice daily for a 5-day period.

FIG. 10A presents results of an in vitro experiment in which leuprolide acetate was administered to cells of the ACHN kidney cancer cell line twice daily for a 5-day period.

FIG. 10B presents results of an in vitro experiment in which leuprolide acetate was administered to cells of the ACHN kidney cancer cell line twice daily for a 5-day period.

FIG. 10C presents results of an in vitro experiment in which leuprolide acetate was administered to cells of the ACHN kidney cancer cell line twice daily for a 5-day period.

FIG. 11A presents results of an in vitro experiment in which leuprolide acetate was administered to cells of the 786-O kidney cancer cell line twice daily for a 5-day period.

FIG. 11B presents results of an in vitro experiment in which leuprolide acetate was administered to cells of the 786-O kidney cancer cell line twice daily for a 5-day period.

FIG. 11C presents results of an in vitro experiment in which leuprolide acetate was administered to cells of the 786-O kidney cancer cell line twice daily for a 5-day period.

FIG. 12 presents results of an in vitro experiment in which leuprolide acetate was administered to cells of the HT-29 colon cancer cell line twice daily for a 5-day period.

FIG. 13A presents results of an in vitro experiment in which leuprolide acetate was administered to cells of the HCT-116 colon cancer cell line twice daily for a 5-day period.

FIG. 13B presents results of a duplicate in vitro experiment in which leuprolide acetate was administered to cells of the HCT-116 colon cancer cell line twice daily for a 5-day period.

FIG. 13C presents results of a triplicate in vitro experiment in which leuprolide acetate was administered to cells of the HCT-116 colon cancer cell line twice daily for a 5-day period.

FIG. 14 presents results of an in vitro experiment in which leuprolide acetate was administered to cells of the HS294T malignant melanoma cancer cell line twice daily for a 5-day period.

FIG. 15 presents results of an in vitro experiment in which leuprolide acetate was administered to cells of the RPMI 7951 malignant melanoma cancer cell line twice daily for a 5-day period.

FIG. 16 presents results of a pharmacokinetic study in which male and female dogs were either injected intramuscularly with a leuprolide depot formulation or implanted subcutaneously with a leuprolide implant formulation.

FIG. 17 presents tumor growth data from an experiment in which human LN229 brain cancer cells were injected as xenografts into nude mice that were concurrently treated with placebo or leuprolide implants. Large tumors: ≧6000 mm³, medium tumors: 2000-6000 mm³, and small tumors: ≦2000 mm³.

FIG. 18A presents tumor growth data from an experiment in which human U118-MG brain cancer cells were injected as xenografts into nude mice that were treated with placebo or leuprolide implants one week prior to the injection. Large tumors: ≧4000 mm³, medium tumors: 2000-4000 mm³, and small tumors: ≦2000 mm³.

FIG. 18B presents tumor growth data from an experiment in which human U118-MG brain cancer cells were injected as xenografts into nude mice that were concurrently treated with placebo or leuprolide implants. Large tumors: ≧2000 mm³, medium tumors: 1000-2000 mm³, and small tumors: ≦1000 mm³.

FIG. 19A presents tumor growth data from an experiment in which human U87-MG brain cancer cells were injected as xenografts into nude mice that were treated with placebo or leuprolide implants one month prior to the injection. Large tumors: ≧1000 mm³, small tumors: ≦1000 mm³.

FIG. 19B presents tumor growth data from an experiment in which human U87-MG brain cancer cells were injected as xenografts into nude mice that were concurrently treated with placebo or leuprolide implants. Large tumors: ≧2000 mm³, small tumors: ≦2000 mm³.

FIG. 20 presents tumor growth data from an experiment in which human CWR22 prostate cancer cells were injected as xenografts into nude mice that were treated with placebo or leuprolide implants eight days prior to the injection.

FIG. 21 presents tumor growth data from an experiment in which human LNCaP-C42 prostate cancer cells were injected as xenografts into nude mice that were treated with placebo or leuprolide implants twelve days prior to the injection. Large tumors: initial tumor volume>100 mm³; and small tumors: initial tumor volume<100 mm³.

FIG. 22 presents tumor growth data from an experiment in which human HPAC pancreatic cancer cells were injected as xenografts into nude mice that were treated with placebo or leuprolide implants seven days prior to the injection. Large tumors: ≧1500 mm³, small tumors: ≦1500 mm³, extra small tumors: ≦100 mm³.

FIG. 23 presents tumor growth data from an experiment in which human PANC 10.05 pancreatic cancer cells were injected as xenografts into nude mice that were treated with placebo or leuprolide implants seven days prior to the injection. Large tumors: ≧500 mm³, small tumors≦500 mm³.

FIG. 24 presents results of protein expression studies to analyze the expression of GnRH receptor I protein in various cancer cell lines.

FIG. 25 presents results of gene expression studies to analyze the expression of GnRH, GnRH receptor 1, βLH, LH receptor, βFSH and FSH receptor in various cancer cell lines.

FIG. 26 presents representative results of gene expression studies in breast and lung cancer cell lines to illustrate data presented in FIG. 25.

FIG. 27 is a schematic representation of the HPG axis.

### SEQUENCE LISTING FREE TEXT

The nucleotide sequences of eighteen DNA primer sequences are presented as SEQ ID NO:1 through SEQ ID NO:18 in the Sequence Listing of the present application. The free text “Artificial primer sequence” appearing under numeric identifier<223> for each listed sequence indicates that the sequence is that of a primer that was artificially synthesized. The protocol for primer synthesis is set out in detail below in the Experimental Design section of the Detailed Description.

### DETAILED DESCRIPTION

The present invention encompasses methods of preventing or treating HPG axis-positive cancers, or preventing or slowing proliferation of such cancer cells, or inhibiting or preventing upregulation of the cell cycle of such cancers by administering an agent that decreases or regulates blood and tissue levels, production, function, or activity of LH or FSH (an “LH/FSH-inhibiting agent”). According to the invention, the LH/FSH-inhibiting agent comprises one or more of GnRH; leuprolide; triptorelin; buserelin; nafarelin; desorelin; histrelin; goserelin; follistatin; a compound that stimulates the production of follistatin; a GnRH antagonist; a GnRH receptor blocker; cetrorelix; abarelix; a vaccine or antibody that stimulates the production of antibodies that inhibit the activity of any of LH, FSH, or GnRH; a vaccine or antibody that stimulates the production of antibodies that block an LH receptor, an FSH receptor, or a GnRH receptor; a compound that regulates expression of an LH or FSH receptor; a compound that regulates post-receptor signaling of an LH or FSH receptor; or a physiologically acceptable analogue, metabolite, precursor, or salt of any of the foregoing LH/FSH-inhibiting agents.

HPG axis-positive cancers that may be prevented or treated according to the present invention with LH/FSH-inhibiting agents include, but are not limited to, the following: prostate, brain (including but not limited to glioblastoma, astrocytoma, medulloblastoma, neuroblastoma, and meningioma), breast, ovary, endometrial, pancreas, lung, malignant melanoma, renal cell carcinoma, hepatocarcinoma, oral carcinoma, laryngeal carcinoma, angiomyxoma, and colon cancer.

Conventionally, the underlying rationale for using hormonal therapy in the treatment of prostate cancer is the suppression of androgens in the bloodstream to concentrations seen with castration. Therefore, according to conventional therapeutic strategies, once this suppression is achieved, there is no reason to continue to escalate doses of such therapies. However, the present invention provides that higher doses, meaning doses that achieve and maintain higher serum or tissue concentrations of GnRH agonists or antagonists, are more effective at treating, mitigating, slowing the progression of, or preventing multiple cancers.

GnRH agonists are the most commonly used type of hormonal therapy for prostate cancer, with leuprolide acetate being an example of a GnRH agonist used in the treatment of prostate cancer. GnRH agonists are analogues of the endogenous GnRH decapeptide with specific amino acid substitutions. Replacement of the GnRH carboxy-terminal glycinamide residue with an ethylamide group increases the affinity of these analogues for the GnRH receptor compared to the endogenous peptide. Many of these analogues also have a longer half-life than endogenous GnRH (Millar R P, Lu Z L, Pawson A J, Flanagan C A, Morgan K, Maudsley S R. Gonadotropin-releasing hormone receptors. Endocrine Reviews 25:235-275, 2004). Administration of such analogues can result in an initial increase in serum gonadotropin concentrations that persists for several days (there is also a corresponding increase in testosterone in men and estrogen in pre-menopausal women). This can be followed by a precipitous decrease in gonadotropins. This decrease is due to the loss of GnRH signaling due to down regulation of pituitary GnRH receptors (Belchetz P E, Plant T M, Nakai Y, Keogh E J, Knobil E. Hypophysial responses to continuous and intermittent delivery of hypothalamic gonadotropin-releasing hormone. Science 202:631-633, 1978). This is thought to be secondary to the increased concentration of ligand, the increased affinity of the ligand for the receptor, and the continuous receptor exposure to ligand as opposed to the intermittent exposure that occurs with physiological pulsatile secretion.

According to the present invention, the underlying rationale for treating cancers with hormonal therapy is that abnormal cell division in malignant tissues may be driven or promoted by elevated levels of gonadotropins. By reducing the level of gonadotropins in the serum and tissue of patients with cancers, it may be possible to treat, prevent, delay, or mitigate cancer.

In embodiments of the invention, the blood level, production, function, or activity of LH or FSH is decreased or regulated to be near a target blood level, a target production, a target function, or a target activity of LH or FSH, respectively, occurring at or near the time of greatest reproductive function, which in humans corresponds to from about 18 to about 35 years of age.

In other embodiments of the invention, the blood level, production, function, or activity of LH or FSH is decreased or regulated to be approximately as low as possible without unacceptable adverse side effects. An unacceptable adverse side effect is an adverse side effect that, in the reasonable judgment of one of ordinary skill in the art, has disadvantages that outweigh the advantages of treatment.

In yet other embodiments, the blood level, production, function, or activity of LH or FSH is decreased or regulated to be undetectable or nearly undetectable by conventional means known in the art, meaning less than about 0.7 mIU/mL for both LH and FSH in a clinical laboratory, and lower in a commercial laboratory.

Embodiments of the present invention include administration of one or more LH/FSH-inhibiting agents that can be used to decrease or regulate the blood level, production, function, or activity of LH or FSH. In certain embodiments of the invention, GnRH or a GnRH analogue can be administered to decrease or regulate the tissue or blood level, production, function, or activity of LH or FSH. Studies have shown that increased levels of GnRH or its analogues will result in significant decreases in LH and FSH levels. (Thorner M O, et al., The anterior pituitary, in Williams Textbook of Endocrinology 9^{th} edition, eds. Wilson J D, Foster D W, Kronenberg H, Larsen P R, 269, W.B. Saunders Company, Philadelphia, Pa. (1998)). For example, leuprolide, a GnRH analogue, has been shown to increase pituitary secretion of LH and FSH for several days after initial administration. (Mazzei T, et al., Pharmacokinetics, endocrine and antitumor effects of leuprolide depot (TAP-144-SR) in Advanced Prostatic Cancer: A Dose Response Evaluation, Drugs in Experimental and Clinical Research, 15:373-387 (1989)). Thereafter, pituitary GnRH receptors are down-regulated, resulting in a significant decrease in LH and FSH secretion. (Mazzei T, et al., Human pharmacokinetic and pharmacodynamic profiles of leuprorelin acetate depot in prostatic cancer patients, Journal of Internal Medicine Research, 18(suppl):42-56 (1990)).

Examples of GnRH analogues that are useful in the present invention include leuprolide, triptorelin, buserelin, nafarelin, desorelin, histrelin, and goserelin. Other LH/FSH-inhibiting agents that can be used according to the invention include GnRH antagonists, GnRH receptor blockers, such as cetrorelix and abarelix, and LH or FSH receptor blockers. Currently approved GnRH agonists and antagonists, dosage levels, and plasma/serum levels of active medication (according to package inserts and prescribing information) are as follows: LUPRON® DEPOT 3.75 mg 1 month injection gives a mean plasma leuprolide concentration of 4.6-10.2 ng/ml at 4 hours postdosing; LUPRON® DEPOT 7.5 mg 1 month injection gives a mean plasma leuprolide concentration of 20 ng/ml at 4 hours and 0.36 ng/ml at 4 weeks; LUPRON® DEPOT-PED 11.25 mg 1 month injection gives a mean plasma leuprolide concentration of 1.25 ng/ml at 4 weeks; LUPRON® DEPOT-PED 15 mg injection gives a mean plasma leuprolide concentration of 1.59 ng/ml at 4 weeks; LUPRON® DEPOT 22.5 mg 3 month injection gives a mean plasma leuprolide concentration of 48.9 ng/ml at 4 hours and 0.67 ng/ml at 12 weeks; LUPRON® DEPOT 30 mg 4 month injection gives a mean plasma leuprolide concentration of 59.3 ng/ml at 4 hours and 0.3 ng/ml at 16 weeks; VIADUR® 72 mg 12 month implantation gives a mean serum leuprolide concentration of 16.9 ng/ml at 4 hours and 2.4 ng/ml at 24 hours with a 0.9 ng/ml mean serum concentration for 12 months; ELIGARD® 7.5 mg 1 month injection gives a mean serum leuprolide concentration of 25.3 ng/ml at 5 hours and a serum level range of 0.28-2.0 ng/ml for one month; ZOLADEX® 3.6 mg 1 month gives a mean serum concentration of 3 ng/ml at 15 days and 0.5 ng/ml at 30 days; ZOLADEX® 10.8 mg 3 month gives a mean serum concentration of 8 ng/ml on the first day after dosing and thereafter, mean concentrations remain relatively stable in the range of 0.3 to 1 ng/ml to the end of the dosing period; SYNAREL® 200 micrograms gives a peak serum nafarelin concentration range of 0.2-1.4 ng/ml, whereas a single dose of 800 micrograms gives a peak serum concentration range of 0.5 to 5.3 ng/ml; TRELSTAR DEPOT 3.75 mg 1 month gives a mean plasma triptorelin concentration of 28.43 ng/ml at 4 hours and declines to 0.084 ng/ml at 4 weeks; Supprelin 200 μg/ml, 500 μg/ml and 1000 μg/ml for daily injection; SUPREFACT® 6.3 mg 2 month implant or 500 μg every 8 hours for 7 days followed by 200 μg per day; CETROTIDE® 0.25 mg daily or 3.0 mg every 4 days gives a mean plasma cetrorelix concentration of 4.97 ng/ml or 28.5 ng/ml at 4 hours, respectively; PLENAXIS® 100 mg given on days 1, 15, and 28 and every 4 weeks afterward gives a peak concentration of abarelix of 43.4 ng/ml 3 days after dosing and maintains 94% of men studied at castrate levels of androgen (≦50 ng/dL) during the dosing period; ANTAGON 250 μg daily gives a mean plasma ganirelix concentration of 14.8 ng/ml at 4 hours. The GnRH analogues plasma levels listed above are sufficient in prostate cancer patients to achieve the desired endocrine effects of reducing serum androgens to below castrate levels (≦50 ng/dL), resulting in “chemical castration.” The present invention makes use of therapeutically effective amounts of agents or combinations of agents to reduce or suppress local tissue production of hormones of the HPG axis (i.e., effective to cause a paracrine or autocrine effect on the target tissue).

In still other embodiments of the present invention, vaccines or antibodies can be employed to stimulate the production of antibodies that recognize, bind to, block or substantially reduce the activity of LH, FSH, or GnRH. In other embodiments, vaccines or antibodies can be employed to stimulate the production of antibodies that recognize, bind to, or block the receptors for one of LH, FSH, or GnRH. Examples of such vaccines include the Talwar vaccine and the vaccine marketed under the trade name GONADIMMUNE® by Aphton Corporation. Other LH/FSH-inhibiting agents that can be used according to the invention include compounds that regulate expression of LH and FSH receptors and agents that regulate post-receptor signaling of LH and FSH receptors.

In other embodiments of the invention, a sex steroid hormone, such as estrogen, progesterone, or testosterone, or an analogue thereof, may be co-administered with an LH/FSH-inhibiting agent. Through a negative feedback loop, the presence of estrogen, progesterone, or testosterone signals the hypothalamus to decrease the secretion of GnRH. (Gharib S D, et al., Molecular biology of the pituitary gonadotropins, Endocrine Reviews, 11:177-199 (1990); Steiner R A, et al., Regulation of luteinizing hormone pulse frequency and amplitude by testosterone in the adult male rat, Endocrinology, 111:2055-2061 (1982)). The subsequent decrease in GnRH decreases the secretion of LH and FSH. (Thorner M O, et al., The anterior pituitary, in Williams Textbook of Endocrinology, 9th edition, eds. Wilson J D, Foster D W, Kronenberg H, Larsen P R, 269, W.B. Saunders Company, Philadelphia, Pa. (1998)). Thus, according to the present invention, co-administration of estrogen, progesterone, or testosterone further decreases secretion of LH or FSH, and thereby inhibits upregulation of the cell cycle, sometimes with synergistic effects. Moreover, because administration of the LH/FSH-inhibiting agents described above may have the undesired side-effect of reducing the natural production of sex steroids, the present invention also encompasses co-administration of sex steroids in order to replenish the sex steroids.

Since GnRH agonists are peptides, they are generally not amenable to oral administration. Therefore, they are usually administered subcutaneously, intra-muscularly, or via nasal spray. GnRH agonists are highly potent with serum concentrations of less than 1 ng/ml of leuprolide acetate required for testosterone suppression (Fowler, J. E., Flanagan, M., Gleason, D. M., Klimberg, I. W., Gottesman, J. E., and Sharifi, R. (2000) Evaluation of an implant that delivers leuprolide for 1 year for the palliative treatment of prostate cancer. Urol. 55:639-642). Due to their small size and high potency, GnRH agonists are also often considered to be ideal for use in long-acting depot delivery systems. At least ten such products are currently marketed in the United States. The duration of action of these products ranges from one month to one year. Leuprolide acetate has been on the market for close to two decades and continues to demonstrate a favorable side effect profile. Most of the side effects such as hot flashes and osteoporosis can be attributed to the loss of sex steroid production (Stege, R. (2000). Potential side-effects of endocrine treatment of long duration in prostate cancer. Prostate Suppl. 10:38-42).

As demonstrated in the experimental data presented herein, leuprolide treatment of cancer cell lines slows or inhibits growth in a dose dependent manner. Inhibition rates of 10-40% were achieved with the highest dose of leuprolide used in the studies. However, in vivo studies demonstrated better efficacy in the inhibition of cancer cell growth. High, sustained levels of leuprolide slowed the growth of various types of tumor xenografts. The experimental tumor xenograft data demonstrated consistent inhibition or significant slowing of tumor growth when leuprolide implants were used to treat mice bearing tumors. Higher serum levels of leuprolide in these experimental animals were thought to have resulted in higher tissue/tumor levels of leuprolide, which led to better inhibition of growth.

### Experimental Design

The following experiments illustrate the present invention and are not to be construed as limiting the invention described in this specification.

Gene expression studies in cell lines were performed as described below. Cell lines (100,000 cells) were plated in 60 mm dishes in appropriate growth media containing either 1% fetal bovine serum or 1% charcoal-dextran treated fetal bovine serum. Cells were allowed to grow for 5 days and then ribonucleic acid (RNA) was extracted from the cells using Total RNA Isolation Reagent (AbGene, Rochester N.Y.) and following the manufacturer's directions. RNA was extracted from frozen tumors by physically dissociating tumor tissue using metal beads and agitation in a deep well plate. Total RNA Isolation Reagent was used to prepare RNA from dissociated tissues. Total RNA purity and quantity was determined by measuring absorbance at 260 nm and 280 nm using μQuant BioTek Instruments Inc. plate reader and KC Junior software (Winooski, Vt.).

0.5 μg of total RNA was used for the first strand cDNA production using iScript cDNA Synthesis Kit from Bio-Rad (Cat. # 170-8890). The resulting complementary DNA product (one tenth of the volume) was used as template for amplification of human gonadotropin releasing hormone receptor 1 (gnrhr1), human gonadotropin releasing hormone (gnrh), luteinizing hormone beta polypeptide (βlh), luteinizing hormone receptor (lh-r), follicle stimulating hormone beta polypeptide (βfsh), follicle stimulating hormone receptor (fsh-r), and glyceraldehyde-3-phosphate dehydrogenase (gapdh) genes with gene specific primers whose sequences are shown below:

GnRHrec1-For:
(SEQ ID NO: 1)
5′-GACCTTGTCTGGAAAGATCC-3′
GnRHrec1-Rev:
(SEQ ID NO: 2)
5′-CAGGCTGATCACCACCATCA-3′
GAPDH-For:
(SEQ ID NO: 3)
5′-GGGGGAGCCAAAAGGGTCAT-3′
GAPDH-Rev:
(SEQ ID NO: 4)
5′-GCCCCAGCGTCAAAGGTGGA-3′
hsGNRH-For:
(SEQ ID NO: 5)
5′-CCTTATTCTACTGACTTCGTGCGT-3′
hsGNRH-Rev:
(SEQ ID NO: 6)
5′-GGAATATGTGCAACTTGGTGTAAGG-3′
bLH-For:
(SEQ ID NO: 7)
5′-CTGCTGCTGTTGCTGCTGCTG-3′
bLH-Rev:
(SEQ ID NO: 8)
5′-GGTGGTCACAGGTCAAGGGGT-3′
LHRs-For:
(SEQ ID NO: 9)
5′-ACGGCCGGTCTCACTCGACTAT-3′
LHRs-Rev:
(SEQ ID NO: 10)
5′-CGTGGCCTCCAGGAGATTGA-3′
LHRn-For:
(SEQ ID NO: 11)
5′-GATTAAGACATGCCATTCTGA-3′
LHRn-Rev:
(SEQ ID NO: 12)
5′-TTTATTGGTAGCCATTAATTCT-3′
bFSH-For:
(SEQ ID NO: 13)
5′-GACACTCCAGTTTTTCTTCCTTTTC-3′
bFSH-Rev:
(SEQ ID NO: 14)
5′-AGGAATCTGCATGGTGAGCA-3′
FSHRs-For:
(SEQ ID NO: 15)
5′-GACCTCCCGAGGAATGCCAT-3′
FSHRs-Rev:
(SEQ ID NO: 16)
5′-GGTGAGGCTGGCTTCCATGA-3′
FSHRn-For:
(SEQ ID NO: 17)
5′-GACAGAAACTTCATCCACTGTCC-3′
FSHRn-Rev:
(SEQ ID NO: 18)
5′-GCCAGGAATATTAAATTAGATG-3′

The detailed protocol for primer synthesis (an automated procedure) is as follows:

### Materials

Commercial Nucleic Acid Synthesizer
Solution of the four DNA phosphoramidite monomers (bases)
   All the 5′-hydroxyl groups must be blocked with a dimethoxytrityl (DMT) group for all four bases
All phosphorus linkages must be blocked with a cyanoethyl group
Blocking solutions
Reaction chamber and a type of solid support such as controlled pore glass
Dichloroacetic acid or trichloroacetic acid
Tetrazole
Acetic anhydride and N-methylimidazole
Dilute iodine in a water/pyridine/tetrahydrofuran solution
Concentrated ammonia hydroxide
Materials for one desalting method

### Procedure

The solid support was prepared with the desired first base already attached via an ester linkage at the 3′-hydroxyl end. The solid support was then loaded into the reaction column. In each step, the solutions were pumped through the column. The reaction column was attached to the reagent delivery lines and the nucleic acid synthesizer.

Step 1: De-Blocking

The reaction column was washed with either dichloroacetic acid (DCA) or trichloroacetic acid in dichloromethane (DCM) to remove a DMT group from the first base.

Step 2: Base Condensation

Tetrazole activated second monomer base was added to the reaction column. The reaction column was then washed to remove any extra tetrazole, unbound base, and by-products.

Step 3: Capping

The base was capped by undergoing acetylation. Acetic anhydride and N-methylimidazole were added to the reaction column. The reaction column was then washed to remove any extra acetic anhydride or N-methylimidazole.

Step 4: Oxidation

To stabilize the linkage between bases, a solution of dilute iodine in water, pyridine, and tetrahydrofuran was added to the reaction column.

Steps one through four were repeated until all desired bases had been added to the oligonucleotide. Each cycle was approximately 98 or 99% efficient.

After all bases had been added, the oligonucleotide had to be cleaved from the solid support and de-protected before it could be effectively used. This was done by incubating the chain in concentrated ammonia at a high temperature for an extended amount of time.

The last step was desalting, which was done to purify the solution. Desalting removes any species that may interfere with future reactions. The major problematic ingredient in the heterogeneous mixture is the ammonium ion. To filter the solution of the ammonium ions, ethanol precipitation was utilized.

PCR (polymerase chain reaction) was performed with Bio-Rad iTaq polymerase (Cat. # 170-8870) using the following program:

### Stage 1

3 min at 95° C.

2 min at 58° C.

### Stage 2-35 Cycles

20 sec at 95° C.

30 sec at 56° C.

45 sec at 72° C.

### Stage 3

5 min at 72° C.

PCR products were visualized by electrophoresis in 1.1% Agarose TBE gels.

Protein expression studies were carried out as described below. For cell lysate studies, cell lines were plated (about 250,000 cells/plate) in 100 mm dishes in appropriate growth media with 1% fetal bovine serum or 1% charcoal-dextran treated fetal bovine serum. Cells were allowed to grow for 5 days followed by scraping and collection in phosphate buffered solution on ice. Protein lysates were prepared by lysing cell pellets in radioimmunoprecipitation (RIPA) buffer. Cell protein lysates were fractionated using sodium dodecyl sulfate polyacrylamide gel electrophoresis (SDS-PAGE), followed by electroblotting to nylon or nitrocellulose membranes. Western immunoblot analysis was performed using a GnRH receptor I antiserum (rabbit polyclonal antibody raised against amino acids 1-328 representing the full-length GnRH receptor of human origin, Santa Cruz Biotechnology, Santa Cruz, Calif.).

Cell growth assays were performed as described below. CaOV3 (ATCC HTB-75) cells were plated in Dulbecco's modified Eagle's medium with 4 mM L-glutamine, 1.5 g/L sodium bicarbonate, 0.1 mM non-essential amino acids, and 10% fetal bovine serum. H358 (ATCC CRL-5807) cells were plated in RPMI 1640 medium with 2 mM L-glutamine adjusted to contain 1.5 g/L sodium bicarbonate, 4.5 g/L glucose, 10 mM HEPES, 1.0 mM sodium pyruvate and 10% fetal bovine serum. H838 (ATCC CRL-5844) cells were plated in RPMI 1640 medium with 1.5 g/L sodium bicarbonate, 4.5 g/L glucose, 10 mM HEPES, 1.0 mM sodium pyruvate, 10 U/ml insulin and 10% fetal bovine serum. T47D (ATCC HTB-133) cells were plated in Minimum Essential Medium with Earle's Balanced Salt Solution and 4 mM L-glutamine, 1.5 g/L sodium bicarbonate, 1.0 mM sodium pyruvate, and 10% fetal bovine serum. MCF-7 (ATCC HTB-22) cells were plated in Minimum essential medium (Eagle) with 2 mM L-glutamine and Earle's BSS adjusted to contain 1.5 g/L sodium bicarbonate, 0.1 mM non-essential amino acids and 1 mM sodium pyruvate and supplemented with 0.01 mg/ml bovine insulin, 90% and 10% fetal bovine serum. HPAC (ATCC CRL-2119) cells were plated in Dulbecco's modified Eagle's/Ham's F12 medium with 10 ng/ml epidermal growth factor, 0.002 mg/ml insulin, 0.005 mg/ml transferrin, 40 ng/ml hydrocortisone, and 5% fetal bovine serum. Panc (ATCC CRL-2547) cells were plated in RPMI 1640 medium with 1.5 g/L sodium bicarbonate, 4.5 g/L glucose, 10 mM HEPES, 1.0 mM sodium pyruvate, 10 U/ml insulin and 10% fetal bovine serum. MV4-11 (ATCC CRL-9591) cells were plated in Iscove's modified Dulbecco's medium with 4 mM L-glutamine adjusted to contain 1.5 g/L sodium bicarbonate, 90% and 10% fetal bovine serum. ACHN (ATCC CRL-1611) cells were plated in Minimum essential medium (Eagle) with 2 mM L-glutamine and Earle's BSS adjusted to contain 1.5 g/L sodium bicarbonate, 0.1 mM non-essential amino acids, and 1.0 mM sodium pyruvate, 90% and 10% fetal bovine serum. 786-O (ATCC CRL-1932) cells were plated in RPMI 1640 medium with 2 mM L-glutamine adjusted to contain 1.5 g/L sodium bicarbonate, 4.5 g/L glucose, 10 mM HEPES, and 1.0 mM sodium pyruvate, 90% and 10% fetal bovine serum. HT-29 (ATCC HTB-38) cells were plated in McCoy's 5a medium (modified) with 1.5 mM L-glutamine adjusted to contain 2.2 g/L sodium bicarbonate, 90% and 10% fetal bovine serum.

For cell growth assays in a 96 well format, different numbers of cells were plated, depending on the cell line (about 2000 cells/well for CaOV3, about 250 cells/well for 786-O, about 500 cells/well for H838 and H358, about 5000 cells/well for T47D and HPAC, about 1000 cells/well for HPAC, ACHN, HT-29, MCF-7 and about 300,000 cells/well for MV4-11). All cell lines were plated in their respective growth media (supplemented with either 1% regular fetal bovine serum, 1% charcoal/dextran-stripped fetal bovine serum or 0.25% Albumax™ (Invitrogen Corp., Grand Island N.Y.)) and allowed to settle for 24 hours. Leuprolide treatments were commenced shortly after plating the cells. A 10 mM (12.25 mg/ml) solution of leuprolide acetate salt in phosphate buffered saline was prepared and diluted appropriately to obtain the desired final concentrations. Treatment concentrations were 0 M (control), 10⁻¹¹ M (shown as 1.00E-11, 0.012 ng/ml), 10⁻⁹ M (shown as 1.00E-9, 0.0012 μg/ml), 10⁻⁸ M (shown as 1.00E-8, 0.012 μg/ml), 10⁻⁷ M (shown as 1.00E-7, 0.12 μg/ml), and 10⁻⁵ M (shown as 1.00E-5, 12.25 μg/ml). The number of cells in each group was measured by incubating cells with WST-8 (2-(2-methoxy-4-nitrophenyl)-3-(4-nitrophenyl)-5-(2,4-disulfophenyl)-2H-tetrazolium, monosodium salt) which produces a water soluble formazan dye that was detected by measuring optical density (at 450 nm) using a μQuant™ Universal Microplate Spectrophotometer (Bio-Tek® Instruments, Inc., Winooski, Vt.).

For brain and prostate cancer tumor xenograft studies, male or female nude:nude athymic mice from Harlan Sprague Dawley (Indianapolis, Ind.) were used. Mice were anesthetized with Domitor/Ketaset and placed under a warming lamp. LN229 (ATCC CRL-2611) cells were prepared by plating in Dulbecco's modified Eagle's medium with 4 mM L-glutamine adjusted to contain 1.5 g/L sodium bicarbonate and 4.5 g/L glucose, 95%; fetal bovine serum, 5%. U87-MG (ATCC HTB-14) cells were prepared by plating in Minimum essential medium (Eagle) with 2 mM L-glutamine and Earle's BSS adjusted to contain 1.5 g/L sodium bicarbonate, 0.1 mM non-essential amino acids, and 1.0 mM sodium pyruvate, 90%; fetal bovine serum, 10%. U118-MG (ATCC HTB-15) cells were prepared by plating in Dulbecco's modified Eagle's medium with 4 mM L-glutamine adjusted to contain 1.5 g/L sodium bicarbonate and 4.5 g/L glucose, 90%; fetal bovine serum, 10%. CWR22 recurrent prostate cancer cells were prepared as described in Wainstein M A, He F, Robinson D, Kung H-J, Schwartz S, Giaconia J M, Edgehouse N L, Pretlow T P, Bodner D R, Kursh E D, Resnick M I, Seftel A, Pretlow T G. CWR22: Androgen-dependent xenograft model derived from a primary human prostatic carcinoma. Cancer Res. 54:6049-6052, 1994. Briefly, CWR22 tumors growing in nude mice were resected following cervical dislocation of the host animal. Tumors were dissected into 100 mg pieces and placed into a 100 mm³ culture dish with RPMI 1640 medium with 2 mM L-glutamine adjusted to contain 1.5 g/L sodium bicarbonate, 4.5 g/L glucose, 10 mM HEPES, and 1.0 mM sodium pyruvate, 90%; fetal bovine serum, 20%. Tissue pieces were minced for five minutes with sterile scissors and allowed to settle. Cells and tissue pieces in solution were pipetted into a 100 ml glass bottle containing the above culture medium containing 0.1% protease enzyme. This mixture was placed on a stir plate with a stir bar in the bottle and stirred at room temperature for 20 minutes followed by 2 minutes without stirring. The medium containing cells was decanted into a 50 ml culture tube on ice and more culture medium with enzyme was added to the bottle with the tumor tissue. This process was repeated eight times, with the supernatant being collected on ice each time. The final combined supernatants were mixed, cell numbers were determined by counting with a hemacytometer, and an aliquot of cells was subjected to centrifugation at 1200×g for 15 minutes and the supernatant was discarded. The resulting cell pellet was resuspended in an appropriate volume of Matrigel™ (Becton Dickinson) at 4° C., triturated repeatedly through an 18-G needle and 5 ml syringe, followed by repeated trituration through a 22-G needle and 1 ml syringe. 100 μl aliquots of tumor cells were injected through a 22-G needle subcutaneously on the flanks of nude mice.

LNCaP-C42 cells (UroCor, Inc., Oklahoma City, Okla.) were cultured in RPMI 1640 medium with 2 mM L-glutamine adjusted to contain 1.5 g/L sodium bicarbonate, 4.5 g/L glucose, 10 mM HEPES, and 1.0 mM sodium pyruvate, 90%; fetal bovine serum, 10%. Cultured cells were trypsinized, counted and injected in Matrigel (BD Biosciences, Bedford, Md.) or Matrigel:cell growth media (no fetal bovine serum), 1:1 and implants were placed subcutaneously into anesthetized mice. Panc (ATCC CRL-2547) cells were plated in RPMI 1640 medium with 1.5 g/L sodium bicarbonate, 4.5 g/L glucose, 10 mM HEPES, 1.0 mM sodium pyruvate, 10 U/ml insulin and 10% fetal bovine serum. Cultured cells were trypsinized, counted and injected in Matrigel (BD Biosciences, Bedford, Md.) or Matrigel:cell growth media (no fetal bovine serum), 1:1 and implants were placed subcutaneously into anesthetized mice. HPAC (ATCC CRL-2119) cells were plated in Dulbecco's modified Eagle's/Ham's F12 medium with 10 ng/ml epidermal growth factor, 0.002 mg/ml insulin, 0.005 mg/ml transferrin, 40 ng/ml hydrocortisone, and 5% fetal bovine serum. Cultured cells were trypsinized, counted and injected in Matrigel (BD Biosciences, Bedford, Md.) or Matrigel:cell growth media (no fetal bovine serum), 1:1 and implants were placed subcutaneously into anesthetized mice. Tumor measurements were carried out twice weekly using calipers, and length (l) and width (w) were converted to tumor volumes using the following equation: (w²×l)/2. All tumors within one treatment group were used to calculate average tumor volumes±standard deviations. To calculate tumor growth rates, tumor volumes were normalized to the initial tumor volume (V₀). When a single tumor was detectable in a treatment group, that tumor volume was used as V₀ for that treatment group and all tumors measured in that group that formed over time were used to calculate a growth rate (V/V₀). At the end of the experiments, mice were sacrificed by cervical dislocation, and tissues and blood were collected.

The DURIN-Leuprolide 2-month implant used as described below, available from Durect Corporation (Cupertino, Calif.), is a solid formulation comprising approximately 25-30 weight % leuprolide acetate dispersed in a matrix of poly (DL-lactide-co-glycolide). The implant is a cylindrical, opaque rod with nominal dimensions of 1.5 mm (diameter)×2.0 cm (length). The formulation provides 11.25 mg of leuprolide acetate per 2 cm rod, with a substantially uniform release profile. For tumor xenograft studies, the following doses were used: placebo (2 cm of formulation, 0 mg leuprolide acetate); low dose (2 cm of formulation, 11.25 mg leuprolide acetate); medium dose (3 cm of formulation, 16.875 mg leuprolide acetate); high dose (4 cm of formulation, 22.5 mg leuprolide acetate).

### Figure Legends

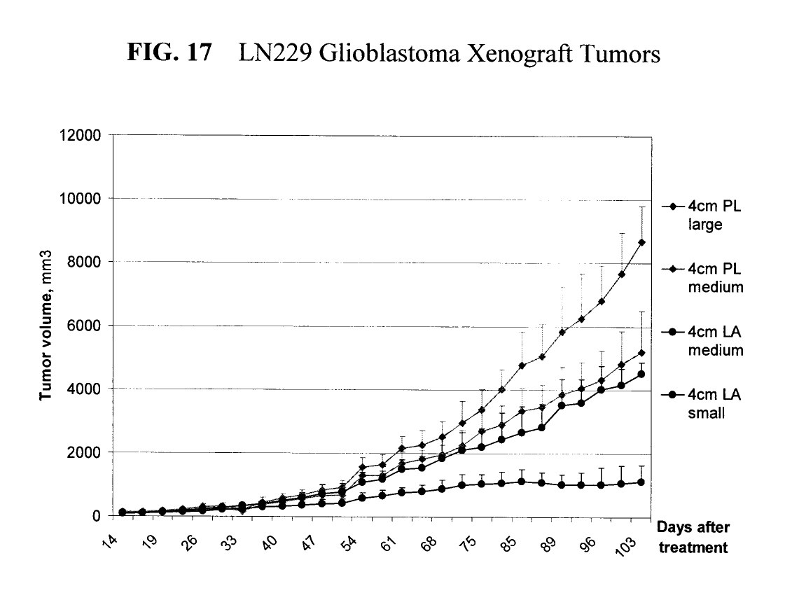

In FIGS. 17-23, “4 cm LA” denotes experimental treatment groups in which the members were implanted with four centimeters of leuprolide rod, and “4 cm PL” denotes experimental placebo groups in which the members were implanted with four centimeters of placebo rod (without leuprolide).

### Experiment 1

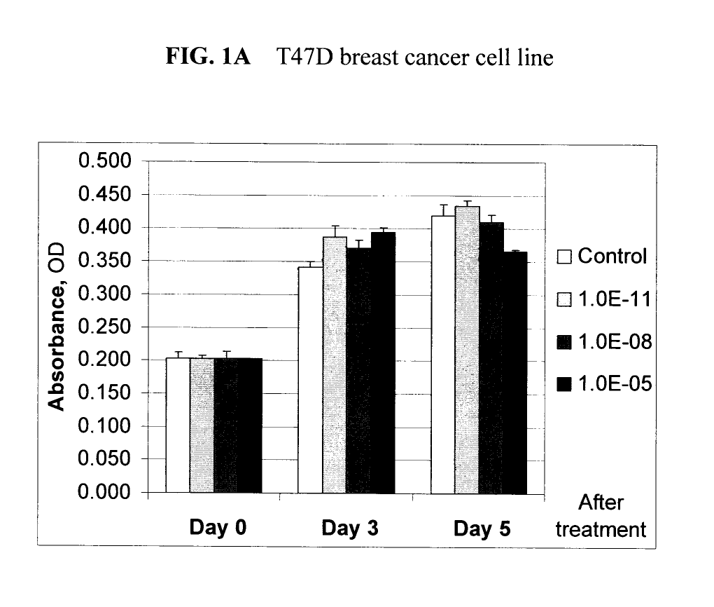

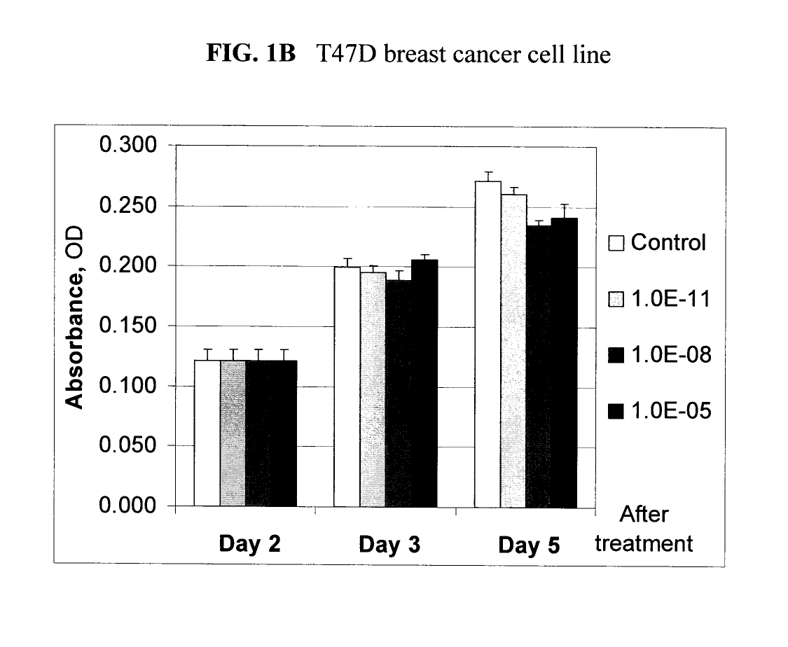

FIGS. 1A and 1B present results of cell growth studies in which the T47D breast cancer cell line was plated in a 96 well plate format and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 0, 3, and 5 or study days 2, 3, and 5.

FIG. 1A presents results of cell growth studies in which about 2500 cells/well were plated in cell culture medium with 1% fetal bovine serum, allowed to grow for 2 days, and then treated twice daily out to day 5.

FIG. 1B presents results of cell growth studies in which about 5000 cells/well were plated in cell culture medium with 1% charcoal-dextran-treated fetal bovine serum. Leuprolide treatments were commenced immediately and performed twice daily out to day 5.

### Experiment 2

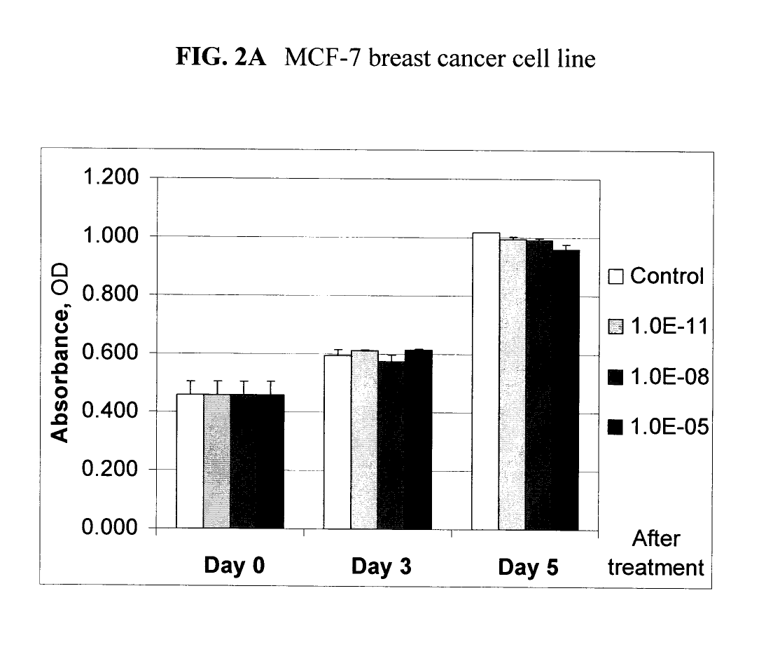

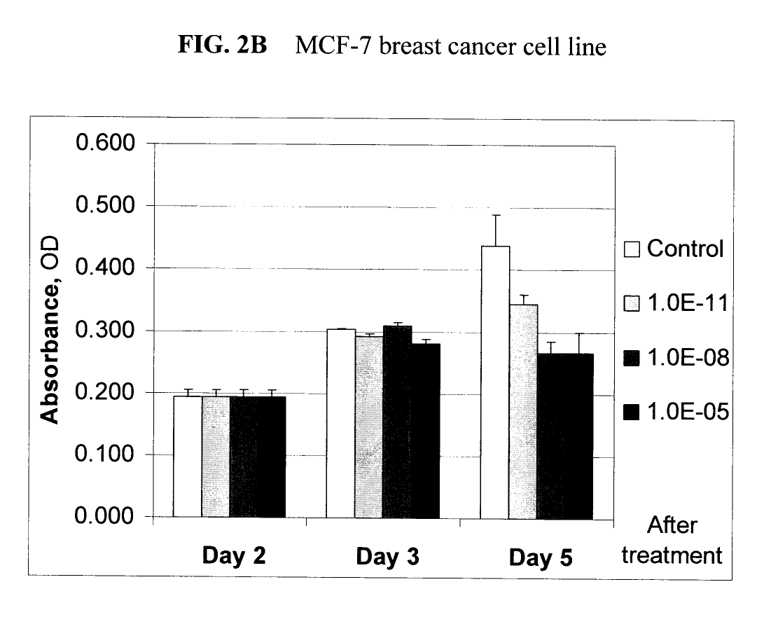

FIGS. 2A and 2B present results of cell growth studies in which the MCF-7 breast cancer cell line was plated in a 96 well plate format and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 0, 3, and 5 or study days 2, 3, and 5.

FIG. 2A presents results of cell growth studies in which about 5000 cells/well were plated in cell culture medium with 1% fetal bovine serum. Leuprolide treatments were commenced immediately and performed twice daily out to day 5.

FIG. 2B presents results of cell growth studies in which about 1000 cells/well were plated in cell culture medium with 1% charcoal-dextran treated fetal bovine serum. Leuprolide treatments were commenced immediately and performed twice daily out to day 5.

### Experiment 3

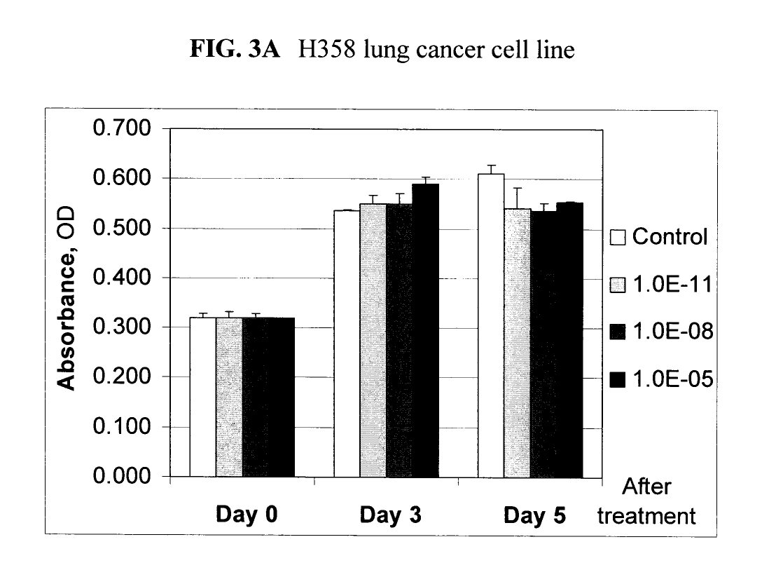

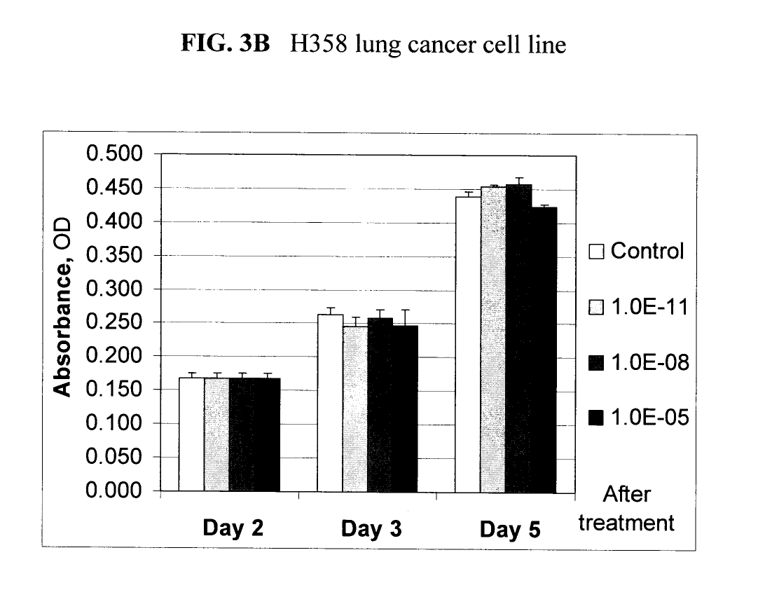

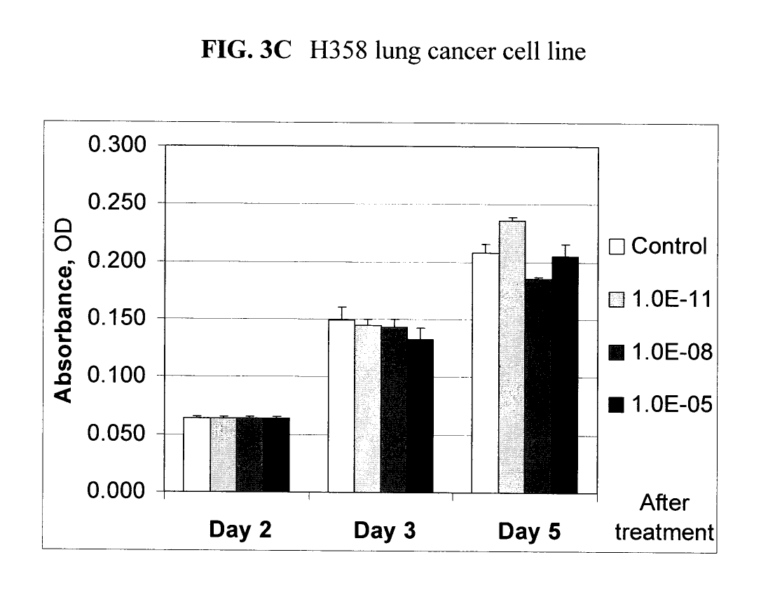

FIGS. 3A, 3B, and 3C present results of cell growth studies in which the H358 non-small cell lung cancer cell line was plated in a 96 well plate format and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 0, 3, and 5 or study days 2, 3, and 5.

FIG. 3A presents results of cell growth studies in which about 1000 cells/well were plated in cell culture medium with 1% fetal bovine serum. Cells were allowed to grow for two days and leuprolide treatments were commenced and performed twice daily out to day 5.

FIG. 3B presents results of cell growth studies in which about 1000 cells/well were plated in cell culture medium with 1% fetal bovine serum. Leuprolide treatments were commenced immediately and performed twice daily out to day 5.

FIG. 3C presents results of cell growth studies in which about 1000 cells/well were plated in cell culture medium with 1% charcoal-dextran treated fetal bovine serum. Leuprolide treatments were commenced immediately and performed twice daily out to day 5.

### Experiment 4

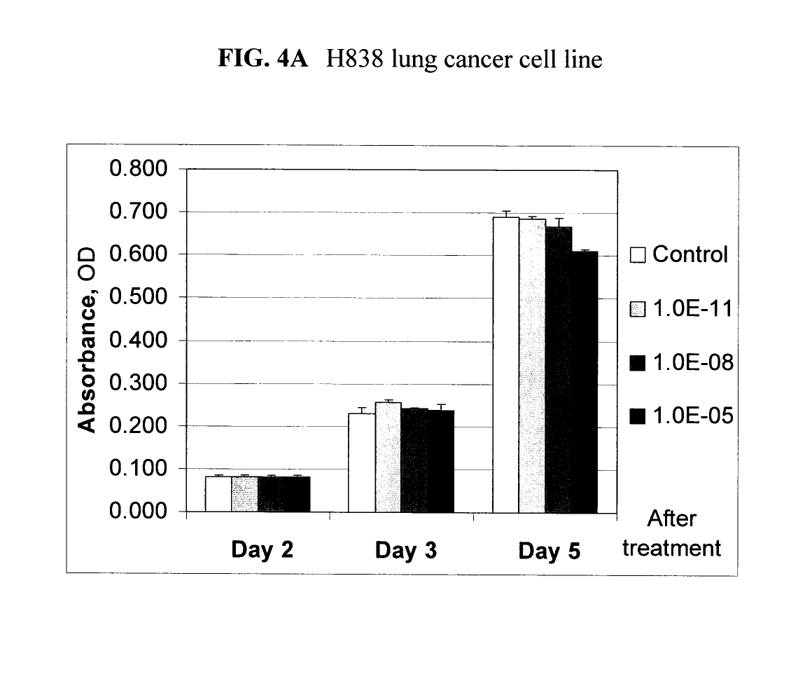

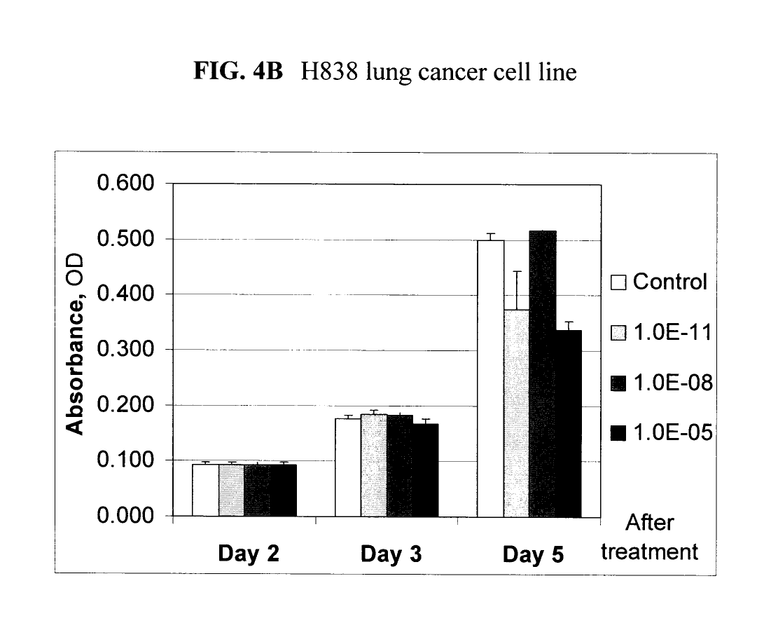

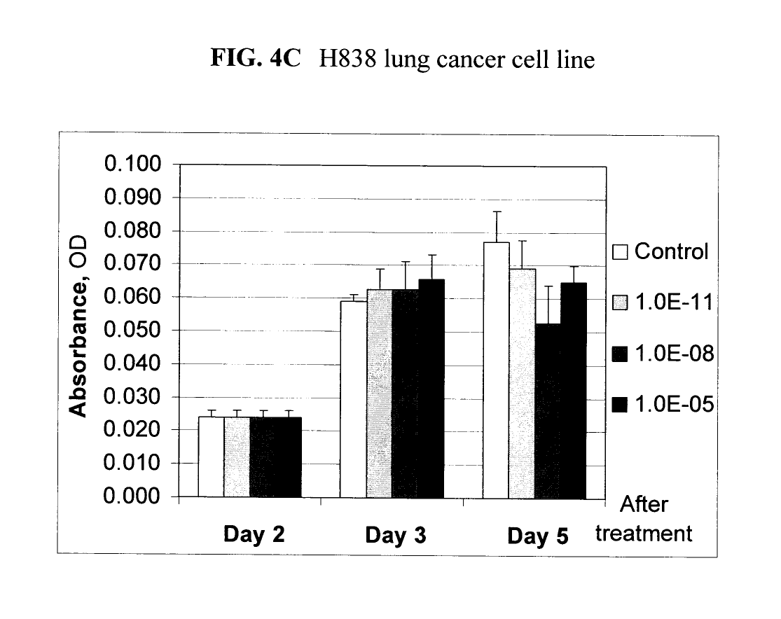

FIGS. 4A, 4B, and 4C present results of cell growth studies in which the H838 non-small cell lung cancer cell line was plated in a 96 well plate format and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 2, 3, and 5.

FIG. 4A presents results of cell growth studies in which about 500 cells/well were plated in cell culture medium with 1% charcoal-dextran treated fetal bovine serum. Leuprolide treatments were commenced immediately and performed twice daily out to day 5.

FIG. 4B presents results of cell growth studies in which about 500 cells/well were plated in cell culture medium with 1% charcoal-dextran treated fetal bovine serum. Leuprolide treatments were commenced immediately and performed twice daily out to day 5.

FIG. 4C presents results of cell growth studies in which about 1000 cells/well were plated in cell culture medium with 0.25% Albumax™ II lipid rich bovine serum albumin. Leuprolide treatments were commenced immediately and performed twice daily out to day 5.

### Experiment 5

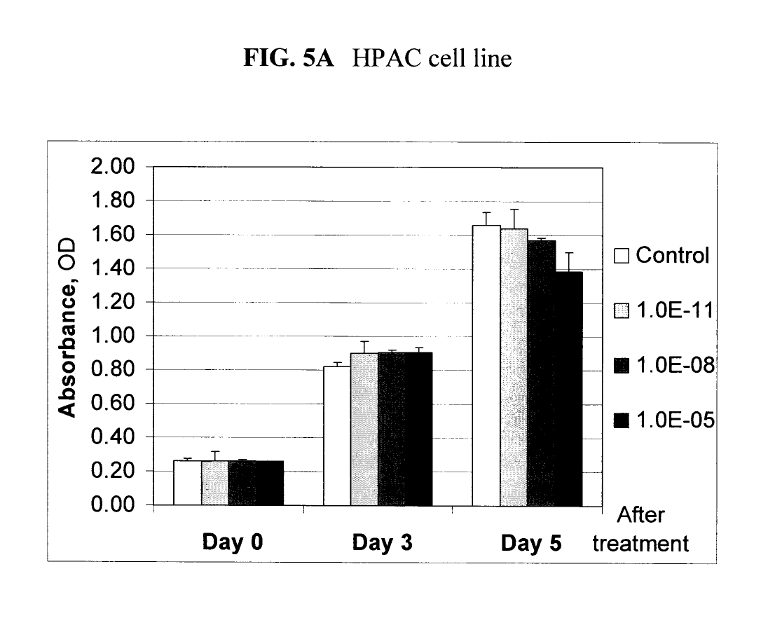

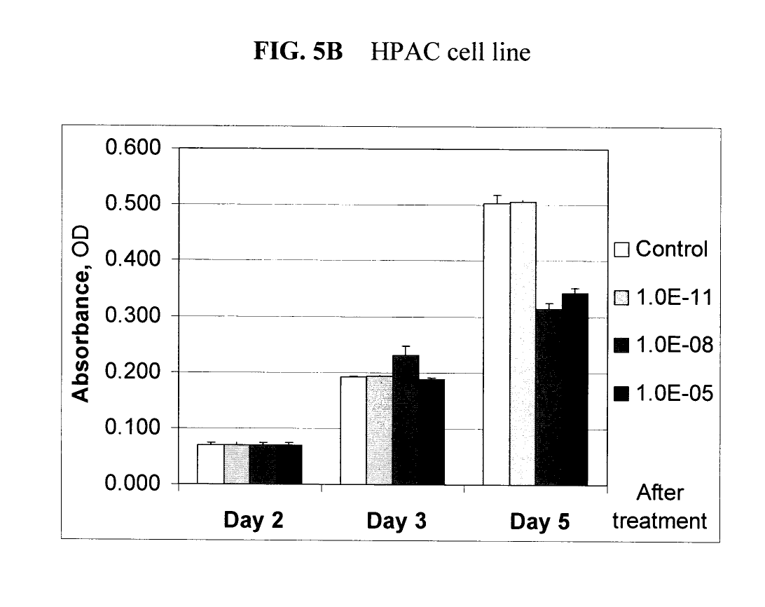

FIGS. 5A and 5B present results of cell growth studies in which the HPAC pancreatic cancer cell line was plated in a 96 well plate format and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 0, 3, and 5 or study days 2, 3, and 5.

FIG. 5A presents results of cell growth studies in which about 2500 cells/well were plated in cell culture medium with 1% fetal bovine serum. Leuprolide treatments were commenced immediately and performed twice daily out to day 5.

FIG. 5B presents results of cell growth studies in which about 5000 cells/well were plated in cell culture medium with 1% charcoal-dextran treated fetal bovine serum. Leuprolide treatments were commenced immediately and performed twice daily out to day 5.

### Experiment 6

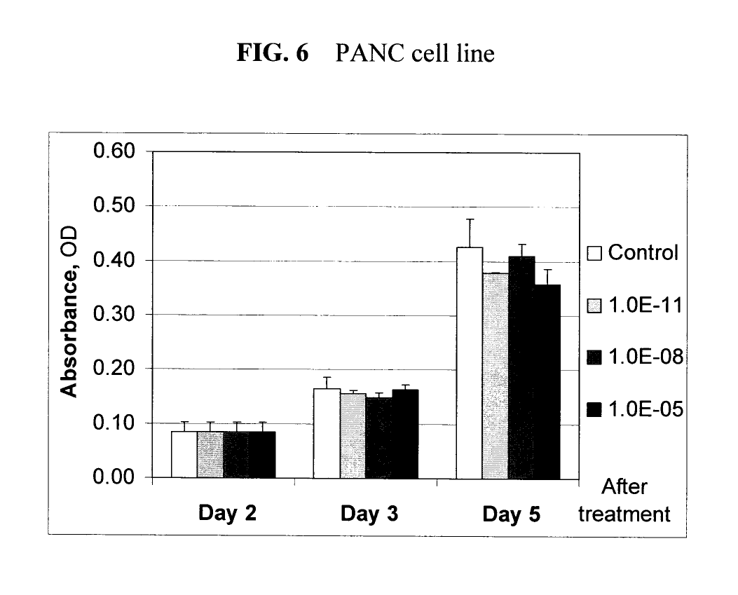

FIG. 6 presents results of cell growth studies in which the PANC pancreatic cancer cell line was plated in a 96 well plate format and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 2, 3, and 5.

FIG. 6 presents results of cell growth studies in which about 5000 cells/well were plated in cell culture medium with 1% charcoal-dextran treated fetal bovine serum. Leuprolide treatments were commenced immediately and performed twice daily out to day 5. FIG. 6 represents the mean absorbances from two independent experiments.

### Experiment 7

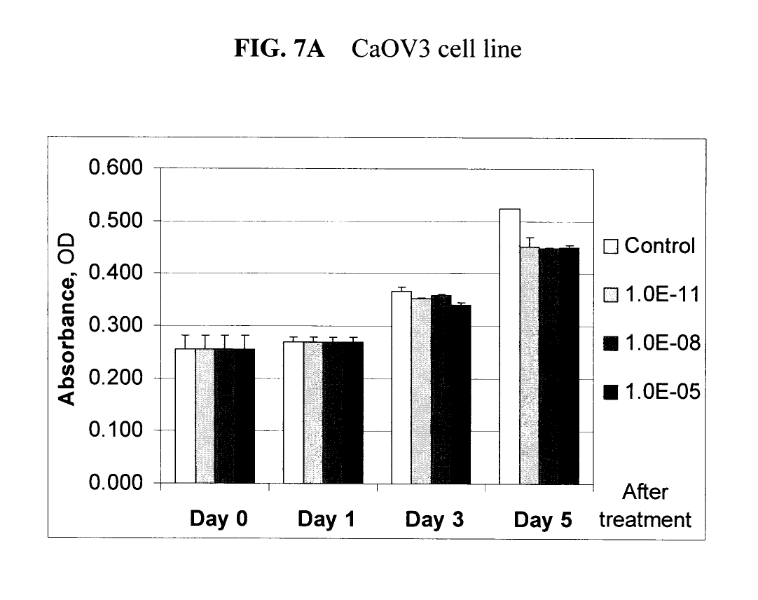

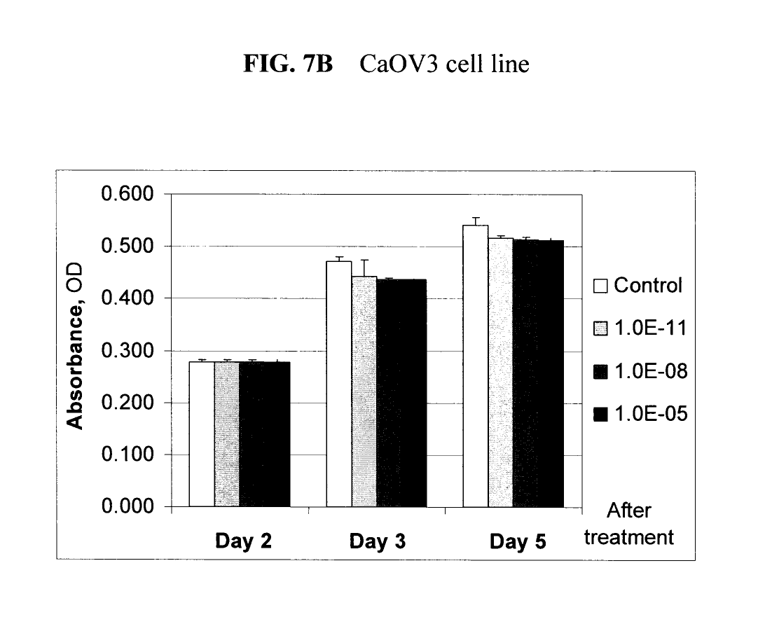

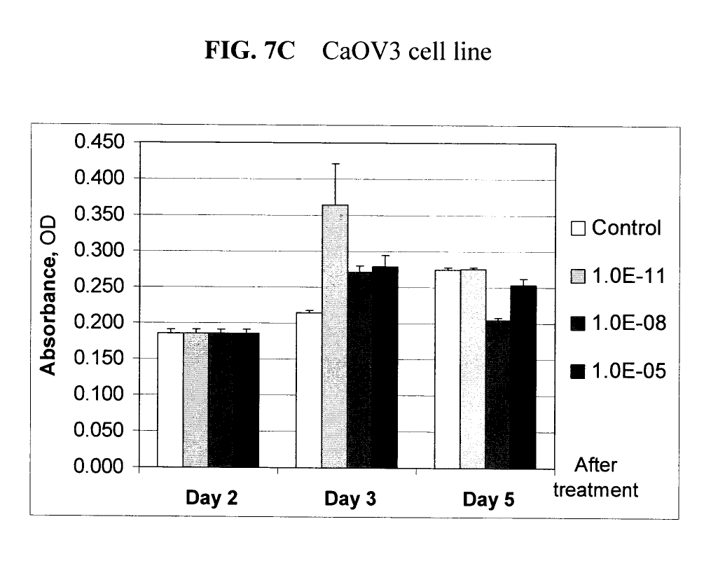

FIGS. 7A, 7B, and 7C present results of cell growth studies in which the CaOV3 ovarian cancer cell line was plated in a 96 well plate format and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 0, 1, 3, and 5 or study days 2, 3, and 5.

FIG. 7A presents results of cell growth studies in which about 2000 cells/well were plated in cell culture medium with 1% fetal bovine serum. Cells were allowed to grow for two days and leuprolide treatments were commenced and performed twice daily out to day 5.

FIG. 7B presents results of cell growth studies in which about 1000 cells/well were plated in cell culture medium with 1% charcoal-dextran treated fetal bovine serum. Leuprolide treatments were commenced immediately and performed twice daily out to day 5.

FIG. 7C presents results of cell growth studies in which about 1000 cells/well were plated in cell culture medium with 1% charcoal-dextran treated fetal bovine serum. Leuprolide treatments were commenced immediately and performed twice daily out to day 5.

### Experiment 8

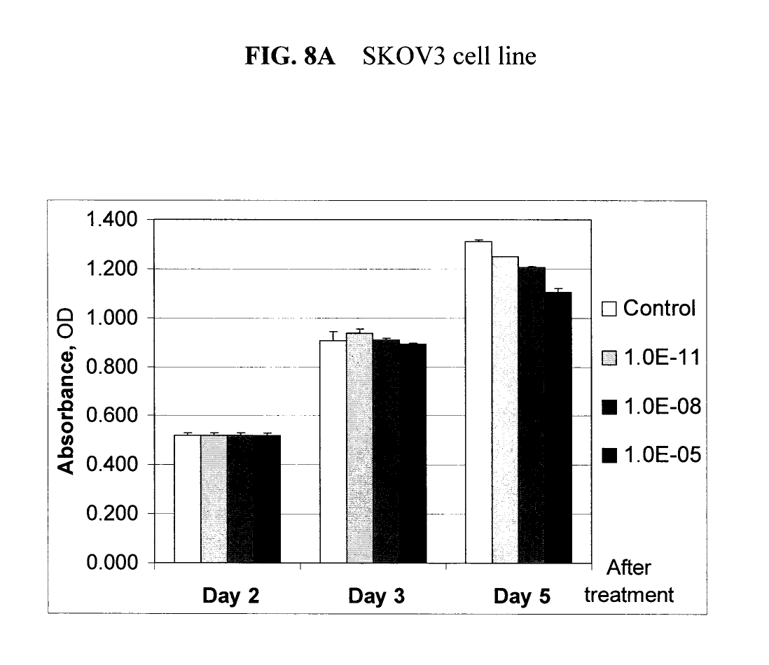

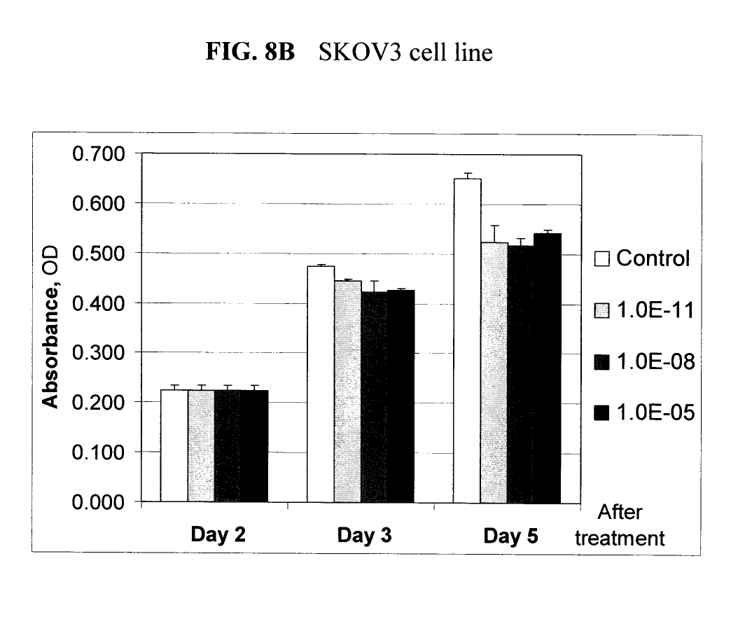

FIGS. 8A and 8B present results of cell growth studies in which the SKOV3 ovarian cancer cell line was plated in a 96 well plate format and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 2, 3, and 5.

FIG. 8A presents results of cell growth studies in which about 1000 cells/well were plated in cell culture medium with 1% charcoal-dextran treated fetal bovine serum. Leuprolide treatments were commenced immediately and performed twice daily out to day 5.

FIG. 8B presents results of cell growth studies in which about 500 cells/well were plated in cell culture medium with 1% charcoal-dextran treated fetal bovine serum. Leuprolide treatments were commenced immediately and performed twice daily out to day 5.

### Experiment 9

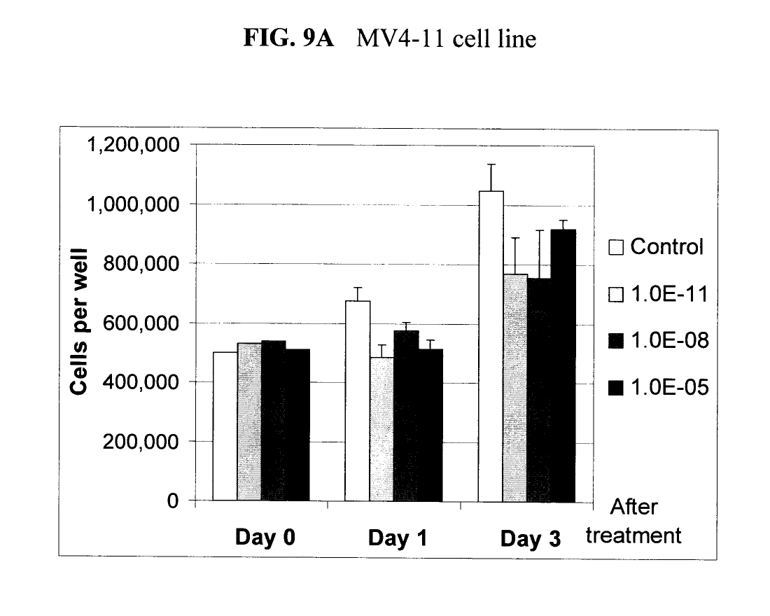

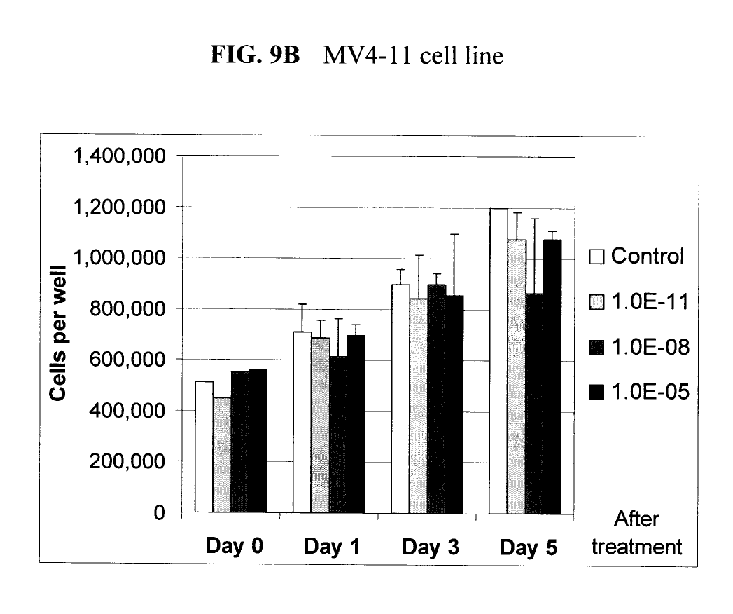

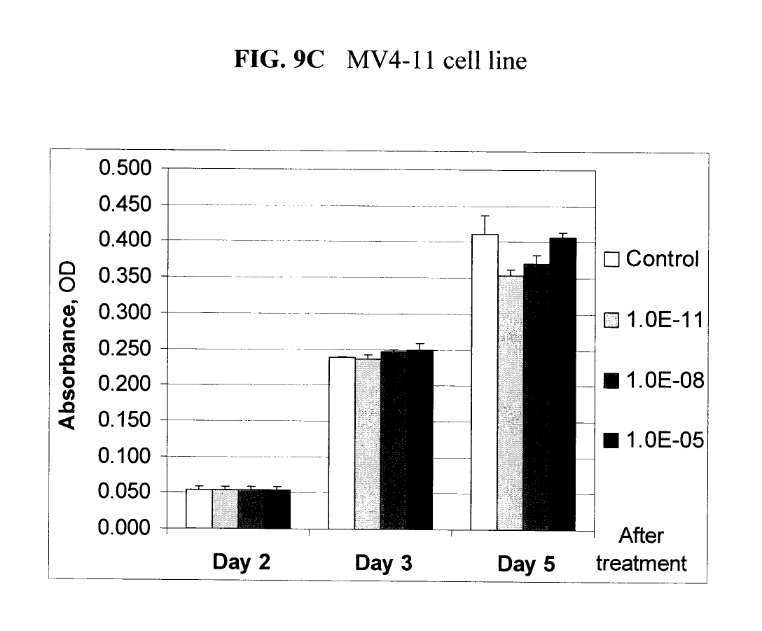

FIGS. 9A, 9B, and 9C present results of cell growth studies in which the MV4-11 leukemia cancer cell line was plated in a 6 well plate format (FIGS. 9A and 9B) or a 96 well format (FIG. 9C) and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 2, 3, and 5.

FIG. 9A presents results of cell growth studies in which about 300,000 cells/well were plated in cell culture medium with 1% charcoal-dextran treated fetal bovine serum. Leuprolide treatments were commenced immediately and performed twice daily out to day 3.

FIG. 9B presents results of cell growth studies in which about 300,000 cells/well were plated in cell culture medium with 1% charcoal-dextran treated fetal bovine serum. Leuprolide treatments were commenced immediately and performed twice daily out to day 5.

FIG. 9C presents results of cell growth studies in which about 1000 cells/well were plated in cell culture medium with 1% charcoal-dextran treated fetal bovine serum. Leuprolide treatments were commenced immediately and performed twice daily out to day 5.

### Experiment 10

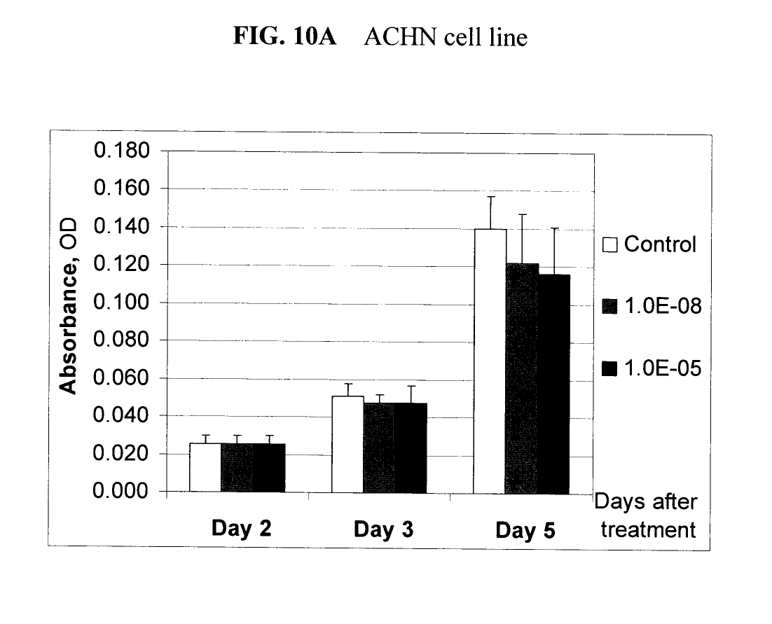

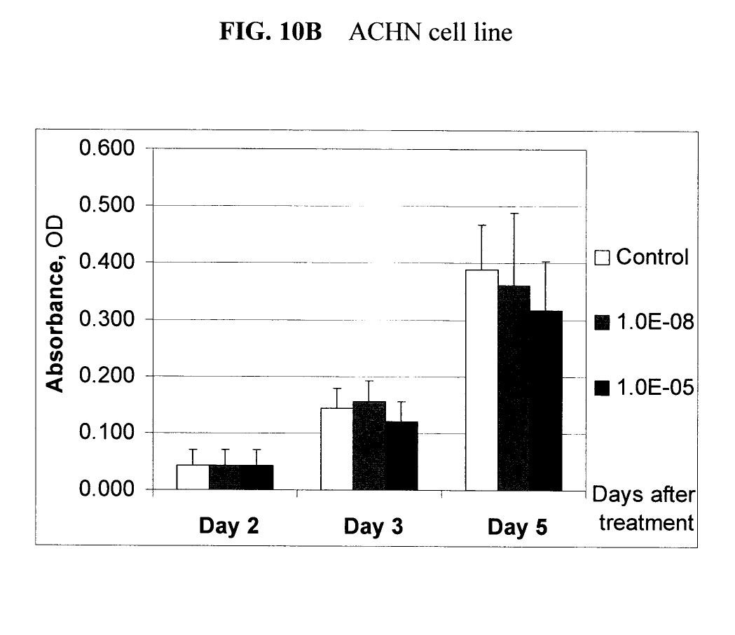

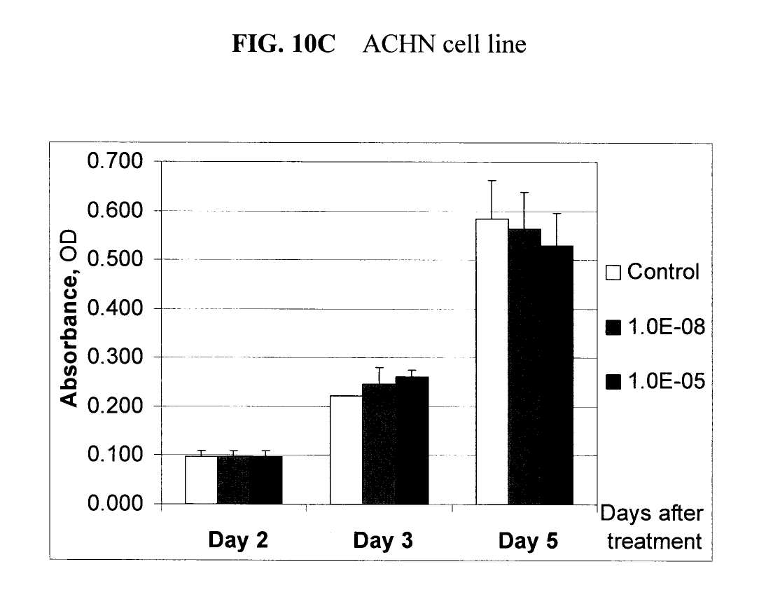

FIGS. 10A, 10B, and 10C present results of cell growth studies in which the ACHN kidney cancer cell line was plated in a 96 well plate format and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 2, 3, and 5.

FIG. 10A presents results of cell growth studies in which about 1000 cells/well were plated in cell culture medium with 1% charcoal-dextran treated fetal bovine serum. Leuprolide treatments were commenced immediately and performed twice daily out to day 5.

FIG. 10B presents results of cell growth studies in which about 500 cells/well were plated in cell culture medium with 1% charcoal-dextran treated fetal bovine serum. Leuprolide treatments were commenced immediately and performed twice daily out to day 5.

FIG. 10C presents results of cell growth studies in which about 250 cells/well were plated in cell culture medium with 1% charcoal-dextran treated fetal bovine serum. Leuprolide treatments were commenced immediately and performed twice daily out to day 5.

### Experiment 11

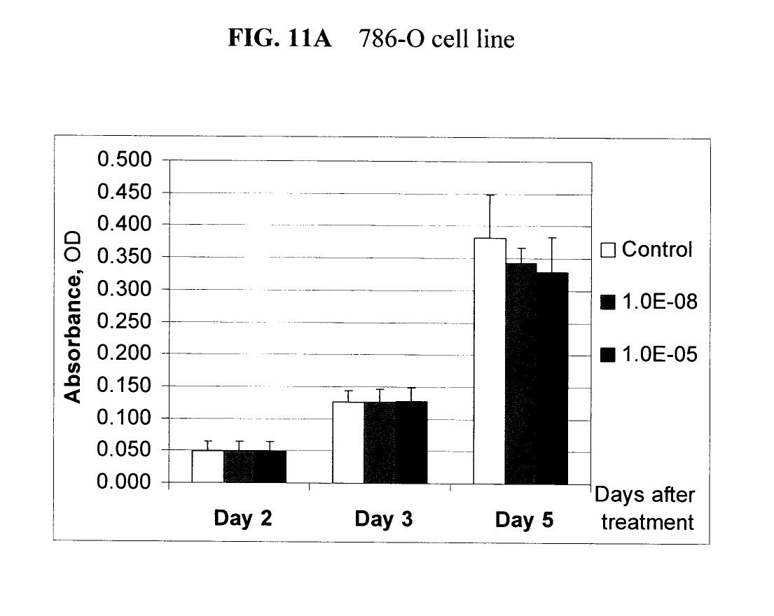

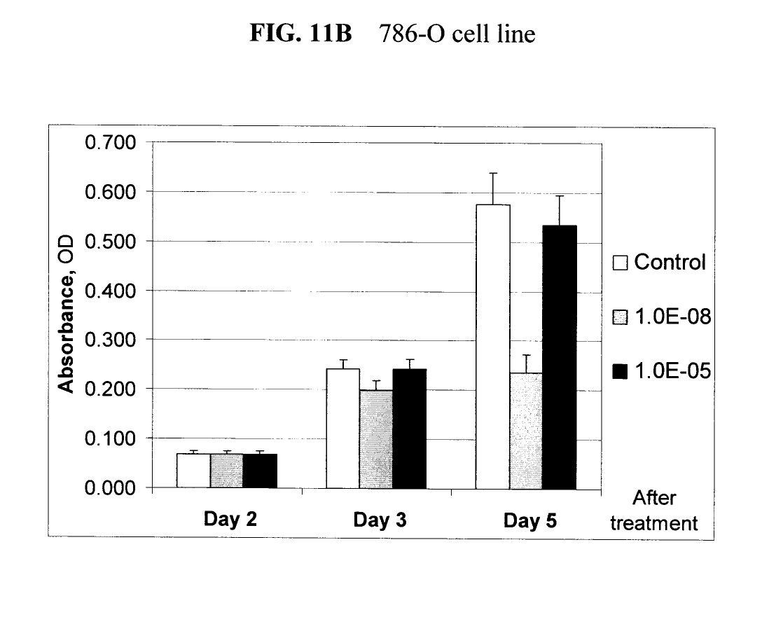

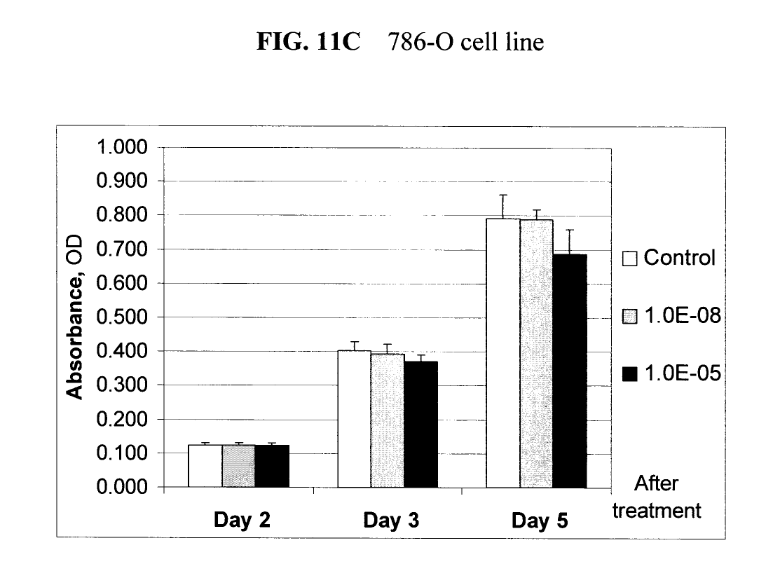

FIGS. 11A, 11B, and 11C present results of cell growth studies in which the 786-O kidney cancer cell line was plated in a 96 well plate format and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 2, 3, and 5.

FIG. 11A presents results of cell growth studies in which about 250 cells/well were plated in cell culture medium with 1% charcoal-dextran treated fetal bovine serum. Leuprolide treatments were commenced immediately and performed twice daily out to day 5.

FIG. 11B presents results of cell growth studies in which about 250 cells/well were plated in cell culture medium with 1% charcoal-dextran treated fetal bovine serum. Leuprolide treatments were commenced immediately and performed twice daily out to day 5.

FIG. 11C presents results of cell growth studies in which about 2500 cells/well were plated in cell culture medium with 1% charcoal-dextran treated fetal bovine serum. Leuprolide treatments were commenced immediately and performed twice daily out to day 5.

### Experiment 12

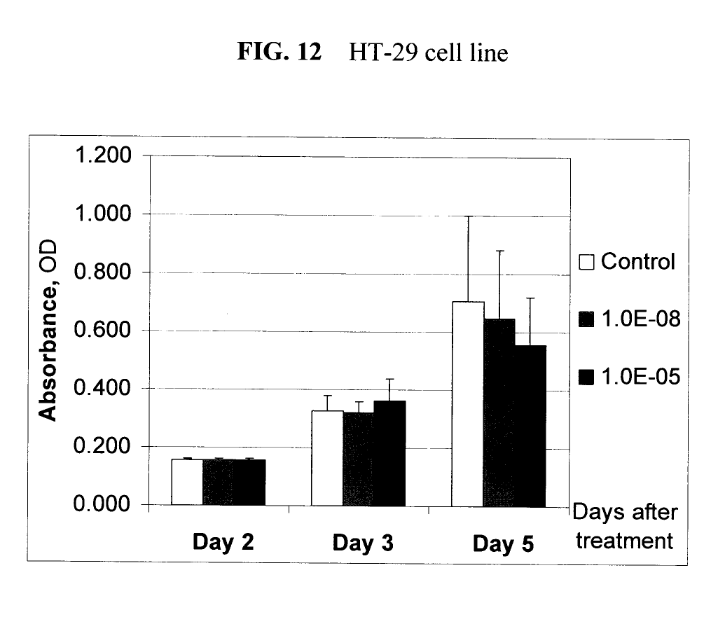

FIG. 12 presents results of cell growth studies in which the HT-29 colon cancer cell line was plated in a 96 well plate format and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 2, 3, and 5.

FIG. 12 presents results of cell growth studies in which about 1000 cells/well were plated in cell culture medium with 1% charcoal-dextran treated fetal bovine serum. Leuprolide treatments were commenced immediately and performed twice daily out to day 5.

### Experiment 13

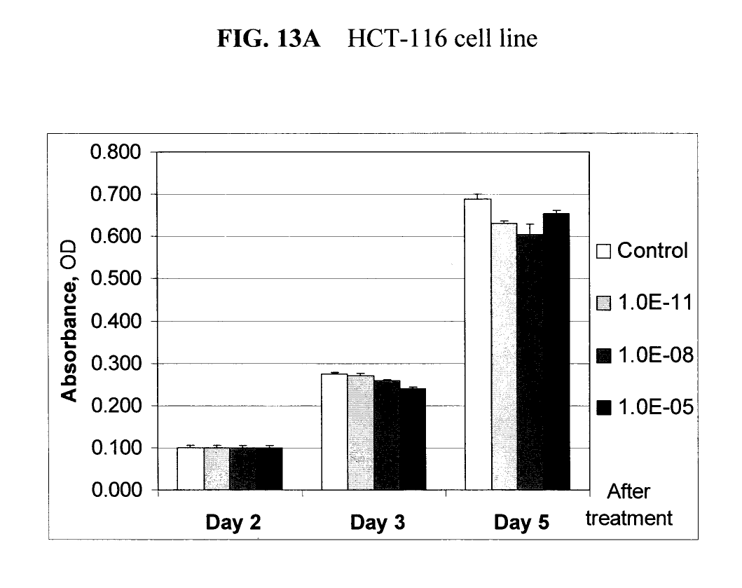

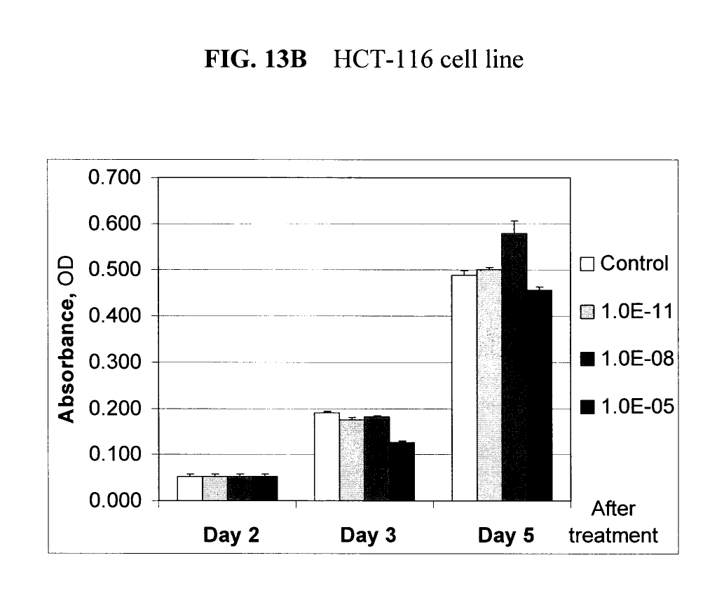

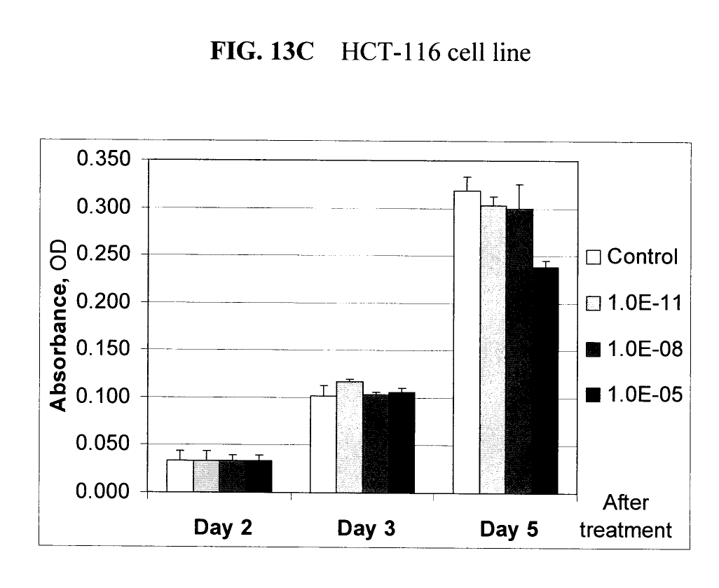

FIGS. 13A, 13B, and 13C present results of cell growth studies in which the HCT-116 colon cancer cell line was plated in a 96 well plate format and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 2, 3, and 5.

FIG. 13A presents results of cell growth studies in which about 1000 cells/well were plated in cell culture medium with 1% charcoal-dextran treated fetal bovine serum. Leuprolide treatments were commenced immediately and performed twice daily out to day 5.

FIG. 13B presents results of cell growth studies in which about 500 cells/well were plated in cell culture medium with 1% charcoal-dextran treated fetal bovine serum. Leuprolide treatments were commenced immediately and performed twice daily out to day 5.

FIG. 13C presents results of cell growth studies in which about 250 cells/well were plated in cell culture medium with 1% charcoal-dextran treated fetal bovine serum. Leuprolide treatments were commenced immediately and performed twice daily out to day 5.

### Experiment 14

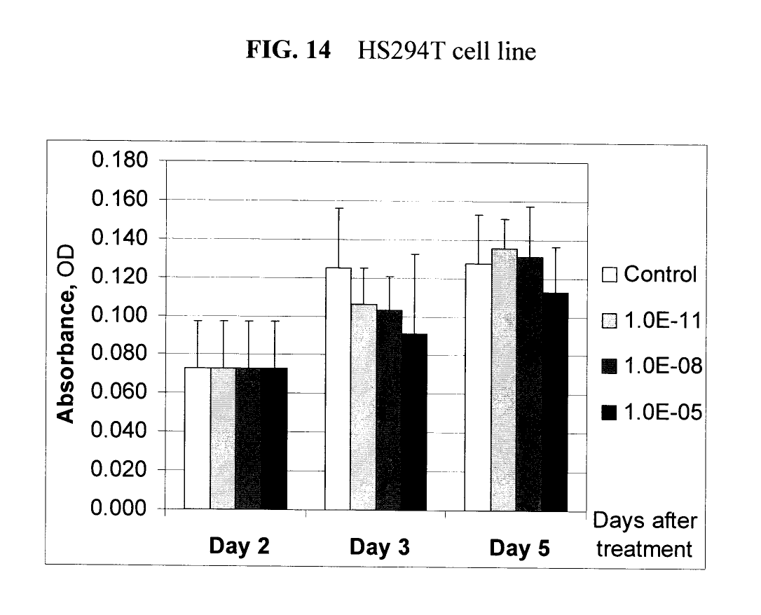

FIG. 14 presents results of cell growth studies in which the HS294T malignant melanoma cancer cell line was plated in a 96 well plate format and treated twice daily for days with leuprolide acetate at doses of 0 M (control), 10 pM (1.0E-11), 10 nM (1.0E-8), or 10 μM (1.0E-5). Absorbance was detected as described above and reflected the number of cells present on study days 2, 3, and 5. FIG. 14 presents results of cell growth studies in which about 100 cells/well were plated in cell culture medium with 1% charcoal-dextran treated fetal bovine serum. Leuprolide treatments were commenced immediately and performed twice daily out to day 5.

### Experiment 15

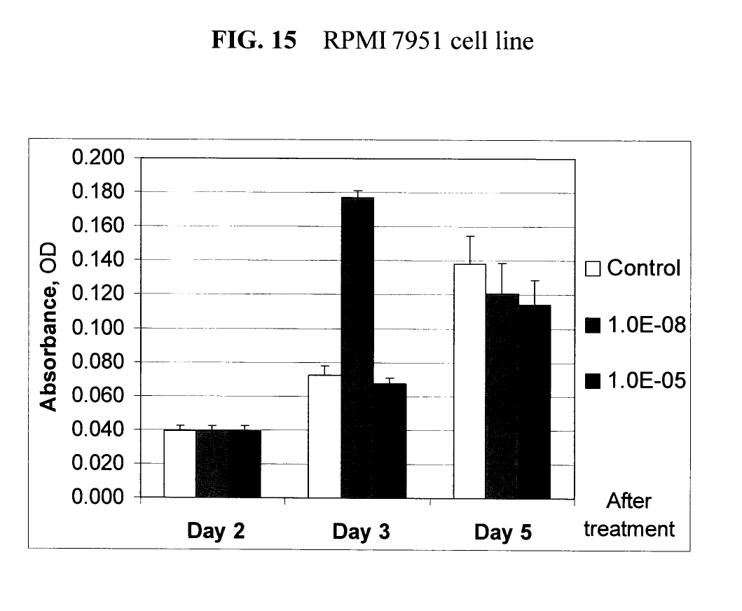

FIG. 15 presents results of cell growth studies in which the RPMI 7951 malignant melanoma cancer cell line was plated in a 96 well plate format and treated twice daily for 5 days with leuprolide acetate at doses of 0 M (control), 10 pM (1.0E-11), 10 nM (1.0E-8), or 10 μM (1.0E-5). Absorbance was detected as described above and reflected the number of cells present on study days 2, 3, and 5. FIG. 15 presents results of cell growth studies in which about 500 cells/well were plated in cell culture medium with 1% charcoal-dextran treated fetal bovine serum. Leuprolide treatments were commenced immediately and performed twice daily out to day 5.

### Experiment 16

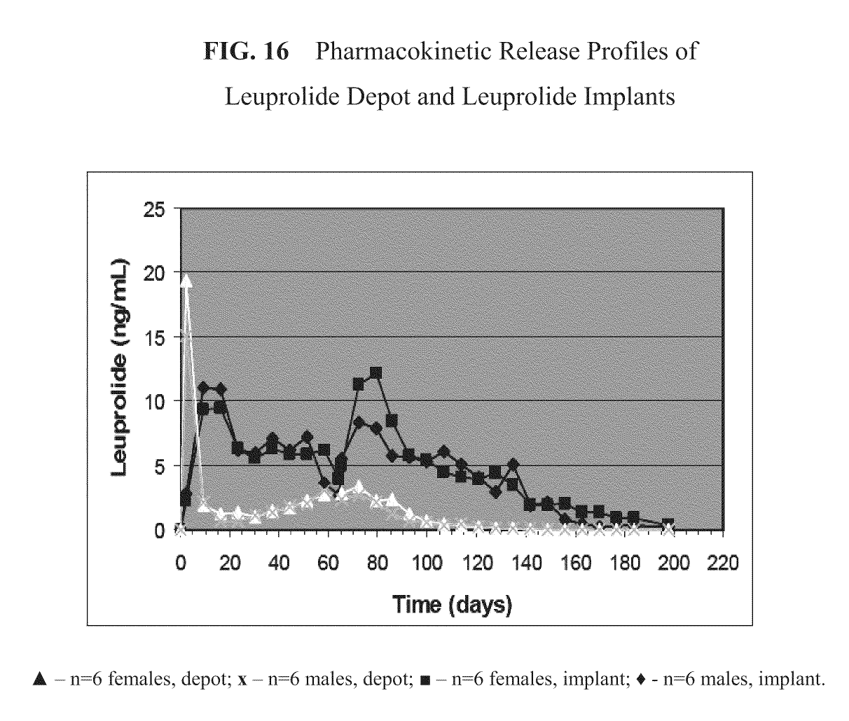

FIG. 16 presents results of a pharmacokinetic study in which male and female dogs were either injected intramuscularly with a leuprolide depot formulation or implanted subcutaneously with a leuprolide implant formulation. Six dogs of each sex were dosed with 60 mg of Lupron Depot® by injection (males—X, females—▴) on day 1. Six dogs of each sex were dosed with single subcutaneous doses (males—▪, females—♦) of 3 DURIN™-Leuprolide 11.3 mg implants (total dose 34 mg) on day 1 and again on day 64. Serum leuprolide levels were determined and plotted against time out to 200 days. Serum leuprolide levels were about 5 to 8 times higher in the DURIN™-Leuprolide treated dogs compared to the Lupron Depot® treated dogs. Higher levels of serum leuprolide sustained over a consistent length of time were thought to have resulted in higher tissue levels of leuprolide sustained over a consistent length of time, which led to inhibition of tumor growth, as demonstrated in FIGS. 17-23.

### Experiment 17

FIG. 17 presents results of an experiment in which about 5×10⁶ cells of the LN229 human glioblastoma brain cancer cell line were injected bilaterally into two groups (one treatment group and a control group), each with four mice. On the same day as the cell injection, a controlled-release leuprolide acetate formulation was implanted into each mouse in the treatment group. Four centimeters of leuprolide rod, providing 22.5 mg of leuprolide, were implanted in each mouse of the treatment group. Four centimeters of placebo rod (without leuprolide) were implanted one week prior to injection into each mouse of the control group.

FIG. 17 presents results of tumor xenograft growth over time in the placebo group and leuprolide implant group. As FIG. 17 shows, tumor volume measurements were commenced on the fourteenth day following injection, when tumors were detectable in all groups. By the 103rd day following injection, large tumors (≧6000 mm³) in the placebo group (n=4) had grown to approximately 8500 mm³ on average, and medium tumors (2000-6000 mm³) in the placebo group (n=4) had grown to approximately 5000 mm³. There were no large tumors in the 4 cm LA treatment group. Medium tumors in the LA group (n=3) had grown to approximately 4500 mm³ and small tumors (n=5) (≦2000 mm³) had grown to 1000 mm³ on average.

### Experiment 18

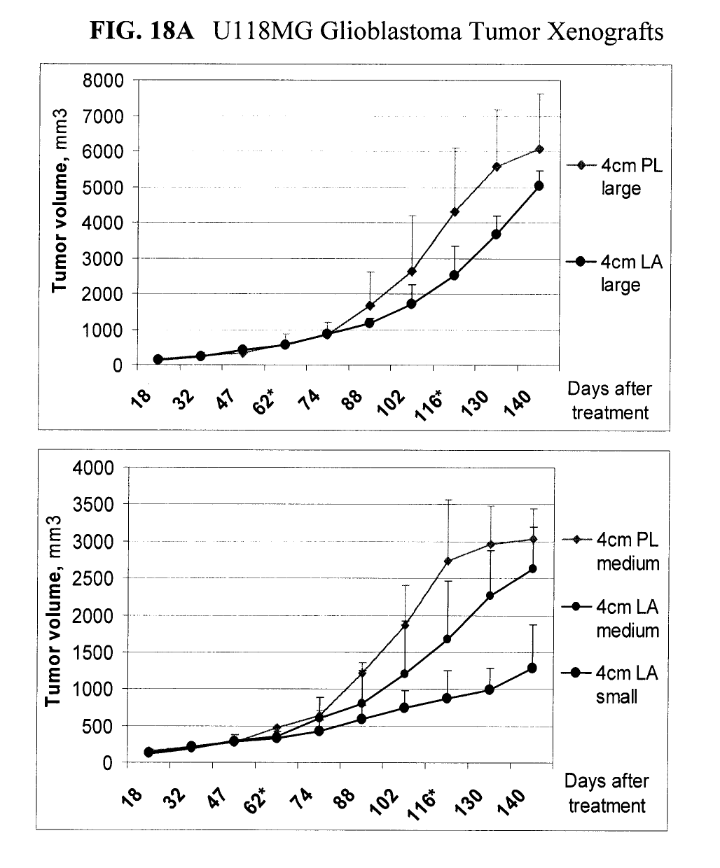

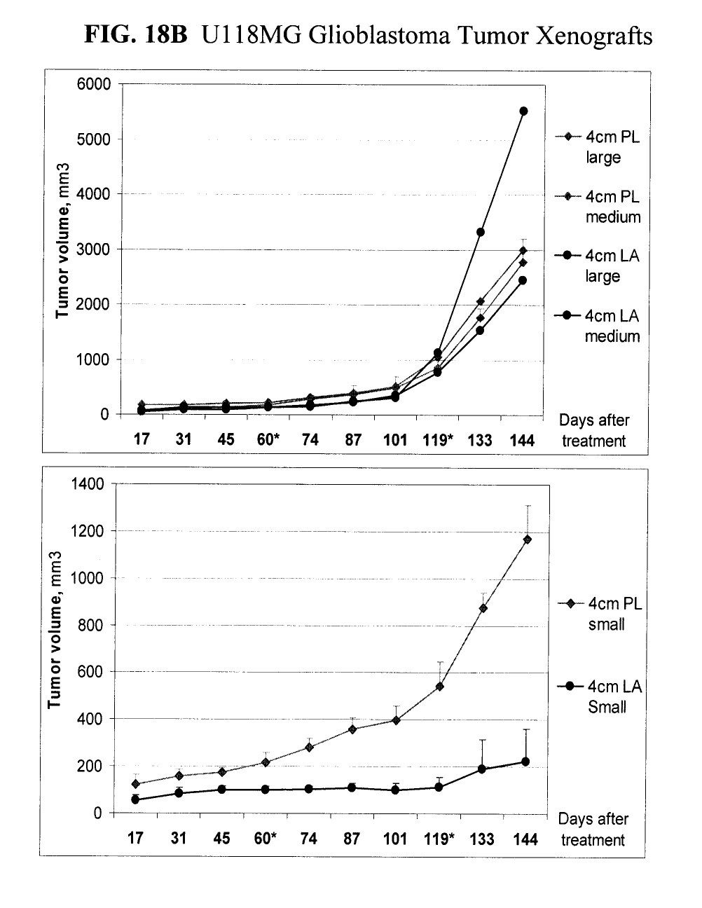

FIGS. 18A and 18B present results of two experiments in each of which about 1×10⁶ cells of the U118-MG human glioblastoma cell line were injected bilaterally into two groups (one treatment group and a control group), each with four mice. Seven days prior to the cell injection (FIG. 18A) or concurrently with cell injection (FIG. 18B), a controlled-release leuprolide acetate formulation was implanted into each mouse in the treatment group. Four centimeters of leuprolide rod, providing 22.5 mg of leuprolide, were implanted in each mouse of the treatment group. Four centimeters of placebo rod (without leuprolide) were implanted one week prior to injection into each mouse of the control group.

FIG. 18A presents results of the first U118 tumor xenograft growth study of tumor xenograft growth over time in the placebo group and the leuprolide implant group. As FIG. 18A shows, tumor measurements were started on day 28 after injection. On day 62, mice were re-dosed with new implants (placebo and leuprolide) and tumor measurements were continued out to 140 days after injection. Due to variation in final tumor volumes, tumors were divided into three groups (large: ≧4000 mm³, medium: 2000-4000 mm³, and small: ≦2000 mm³). At 140 days after injection, the large tumors in the placebo group (n=4) had grown to approximately 6000 mm³ on average, while large tumors in the 4 cm LA group (n=2) had grown to 5000 mm³ on average. The medium tumors in the placebo group (n=2) had grown to approximately 3000 mm³ while the medium tumors in the LA group (n=3) had grown to about 2600 mm³. There were no small tumors in the placebo group, while the small tumors in the 4 cm LA group (n=2) had grown to about 1250 mm³.

FIG. 18B presents results of the second study of U118 tumor xenograft growth over time in the placebo group and the leuprolide implant group. As FIG. 18B shows, tumor measurements were started on day 17 after injection and continued until day 144 after injection. By day 144 after injection, the large tumor (≧2000 mm³) in the 4 cm LA group (n=1) had grown to approximately 5500 mm³ on average, while the large tumor in the placebo group (n=1) had grown to 2800 mm³ on average. Medium tumors (1000-2000 mm³) in the placebo group (n=4) had grown to about 3000 mm³ while medium tumors in the LA group (n=1) had grown to about 2200 mm³. Small tumors in the placebo group (n=3) had grown to about 1200 mm³ while small tumors in the LA group (n=4) had grown to about 200 mm³.

### Experiment 19

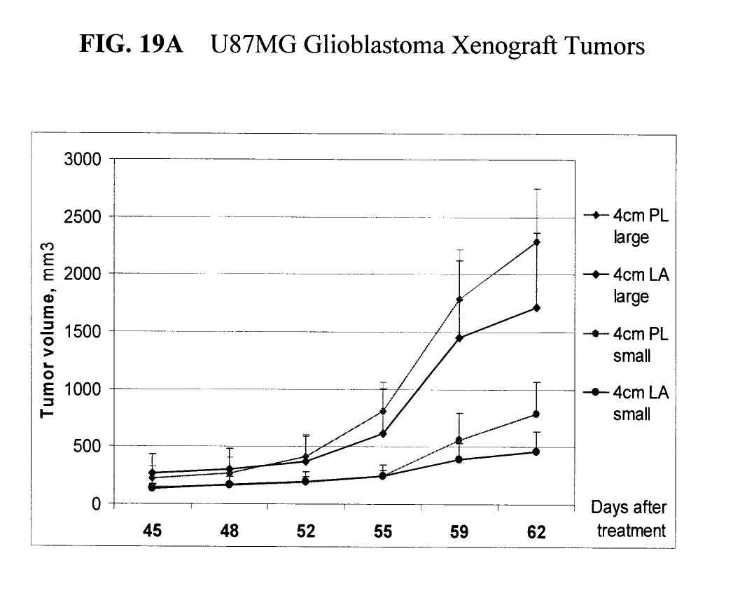

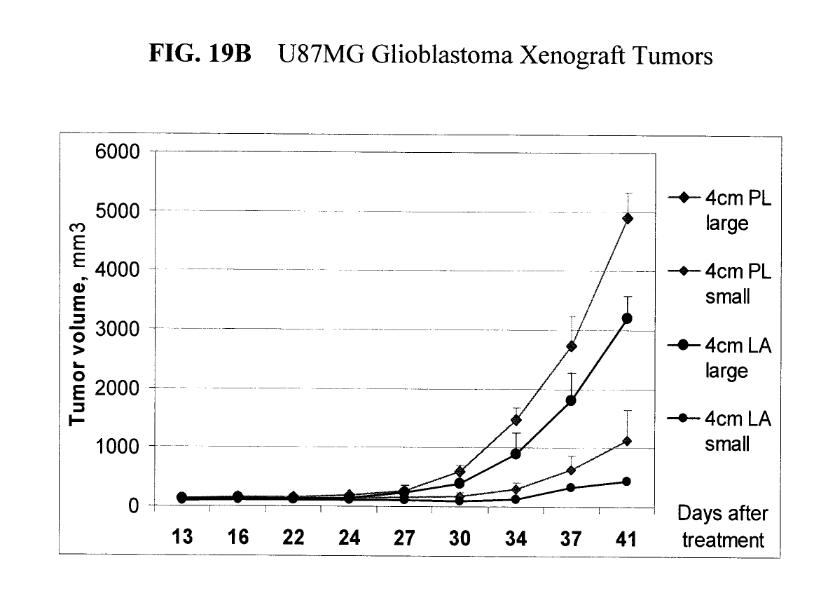

FIG. 19 presents results of two experiments in which about 5.0×10⁶ cells of the U87MG glioblastoma cell line were injected bilaterally into two groups (one treatment group and a control group), each with four mice. One month prior to the cell injection (FIG. 19A) or concurrently with cell injection (FIG. 19B), a controlled-release leuprolide acetate formulation was implanted into each mouse in the treatment group. Four centimeters of leuprolide rod, providing 22.5 mg of leuprolide, were implanted in each mouse of the treatment group. Four centimeters of placebo rod (without leuprolide) were implanted one week prior to injection into each mouse of the control group.

FIG. 19A presents results from a study of U87MG xenograft growth over time in the placebo group and leuprolide implant group. As FIG. 19A shows, tumor measurements were started on day 45 after tumor cell injection and continued until day 62 after injection. By day 62 after injection, the large tumors (≧1000 mm³) in the placebo group (n=5) had grown to approximately 2250 mm³, while the large tumors in the 4 cm LA treatment group (n=3) had grown to about 1700 mm³. Small tumors (≦1000 mm³) in the placebo group (n=3) had grown to about 800 mm³ while small tumors in the 4 cm LA treatment group (n=5) had grown to approximately 475 mm³.

FIG. 19B presents results of tumor xenograft growth over time in the placebo group and the leuprolide implant group. As FIG. 19B shows, tumor measurements were started on day 13 after injection. Due to variation in final tumor volumes, tumors were divided into two groups (large: >2000 mm³ and small: <2000 mm³). By day 41 after injection, large tumors in the placebo group (n=2) had grown to approximately 5000 mm³ on average, while large tumors in the 4 cm LA treatment group (n=7) had grown to 3200 mm³ on average. Small tumors in the placebo group (n=6) had grown to approximately 1100 mm³ on average, while the small tumor in the 4 cm LA treatment group (n=1) had grown to approximately 400 mm³ on average.

### Experiment 20

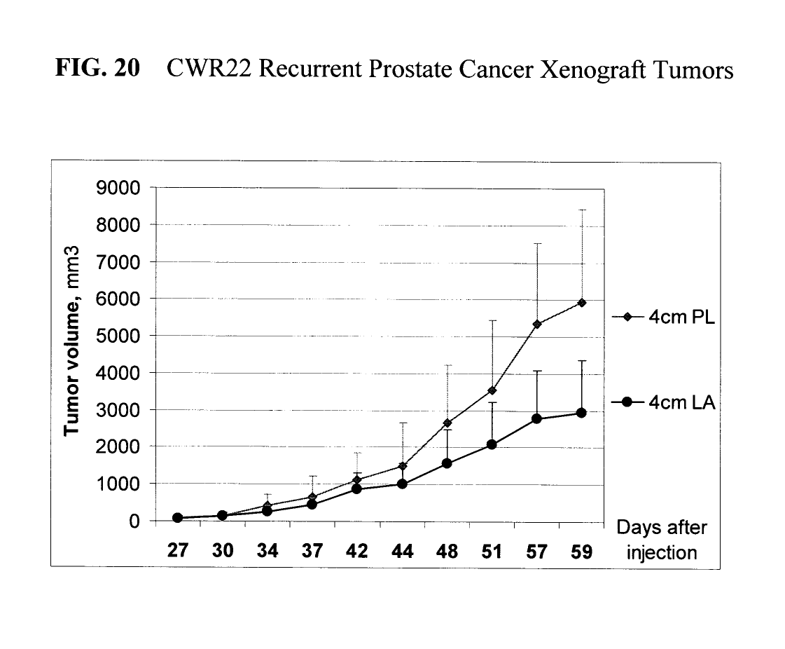

FIG. 20 presents results of an experiment in which about 1.25×10⁶ cells of the CWR22 recurrent prostate cancer xenograft tumor were injected bilaterally into two groups (one treatment group with three mice and a control group with four mice). Eight days prior to the cell injection, a controlled-release leuprolide acetate formulation was implanted into each mouse in the treatment group. Four centimeters of leuprolide rod, providing 22.5 mg of leuprolide, were implanted in each mouse of the treatment group. Four centimeters of placebo rod (without leuprolide) were implanted one week prior to injection into each mouse of the control group.

FIG. 20 presents results of tumor xenograft growth over time in the placebo group and the leuprolide implant group. As FIG. 20 shows, tumor measurements were started on day 27 after injection. By day 59 after injection, tumors in the placebo group (n=8) had grown to approximately 6000 mm³ on average, while tumors in the 4 cm treatment group (n=6) had grown to 3000 mm³ on average.

### Experiment 21

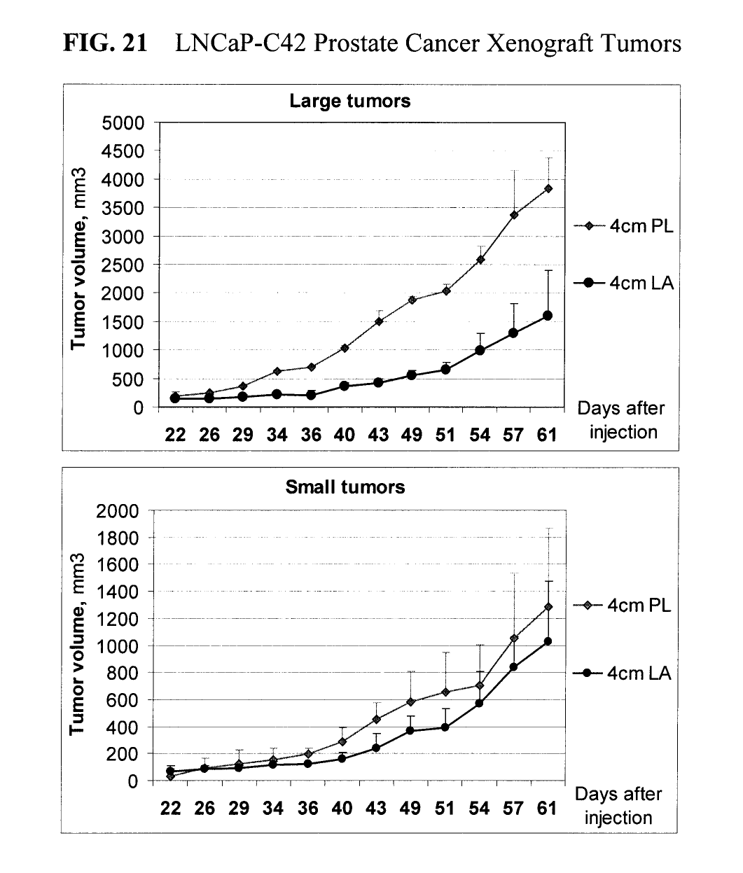

FIG. 21 presents results of an experiment in which about 1.0×10⁶ cells of the LNCaP-C42 prostate cancer xenograft tumor were injected bilaterally into two groups (one treatment group and a control group), each with four mice. Twelve days prior to the cell injection, a controlled-release leuprolide acetate formulation was implanted into each mouse in the treatment group. Four centimeters of leuprolide rod, providing 22.5 mg of leuprolide, were implanted in each mouse of the treatment group. Four centimeters of placebo rod (without leuprolide) were implanted one week prior to injection into each mouse of the control group.

FIG. 21 presents results of tumor xenograft growth over time in the placebo group and the leuprolide implant group. As FIG. 21 shows, tumor measurements were started on day 22 after injection. Tumor data was plotted according to the sizes of the tumors (large tumors: initial tumor volume>100 mm³; and small tumors: initial tumor volume<100 mm³). By day 61 after injection, tumors in the large tumor placebo (PL) group (n=2) had grown to approximately 3800 mm³ on average, while tumors in the large tumor leuprolide (LA) treatment group (n=3) had grown to 1600 mm³ on average. By day 61 after injection, tumors in the small tumor placebo group (n=6) had grown to approximately 1300 mm³ on average, while tumors in the small tumor LA treatment group (n=4) had grown to 1000 mm³ on average.

### Experiment 22

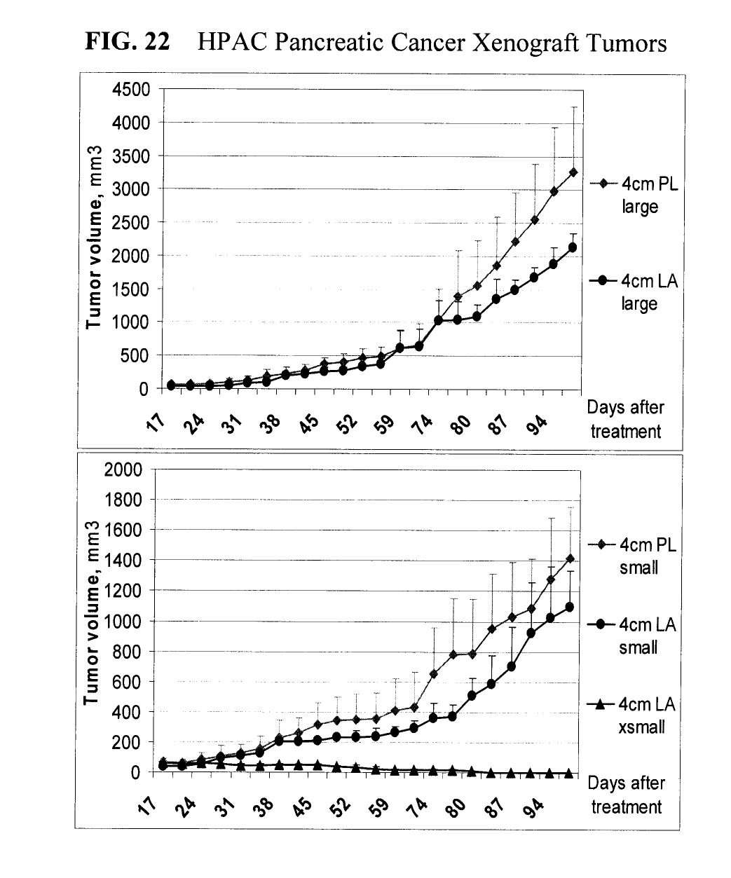

FIG. 22 presents results of an experiment in which about 3.0×10⁶ cells of the HPAC pancreatic cancer cell line were injected bilaterally into two groups (one treatment group and a control group), each with four mice. Seven days prior to the cell injection, a controlled-release leuprolide acetate formulation was implanted into each mouse in the treatment group. Four centimeters of leuprolide rod, providing 22.5 mg of leuprolide, were implanted in each mouse of the treatment group. Four centimeters of placebo rod (without leuprolide) were implanted one week prior to injection into each mouse of the control group.

FIG. 22 presents results of tumor xenograft growth over time in the placebo group and the leuprolide implant group. As FIG. 22 shows, tumor measurements were started on day 17 after injection. Tumor data was plotted according to the sizes of the tumors (large tumors: ≧1500 mm³; small tumors: ≦1500 mm³; and extra small tumors: ≦100 mm³). By day 97 after injection, large tumors in the placebo group (n=5) had grown to approximately 3200 mm³ on average, while large tumors in the LA treatment group (n=2) had grown to 2100 mm³ on average. By day 97 after injection, small tumors in the placebo group (n=3) had grown to approximately 1400 mm³ on average, while small tumors in the LA treatment group (n=2) had grown to 1100 mm³ on average. There were two extra small tumors in the LA treatment group that remained at a constant size of ≦100 mm³.

### Experiment 23

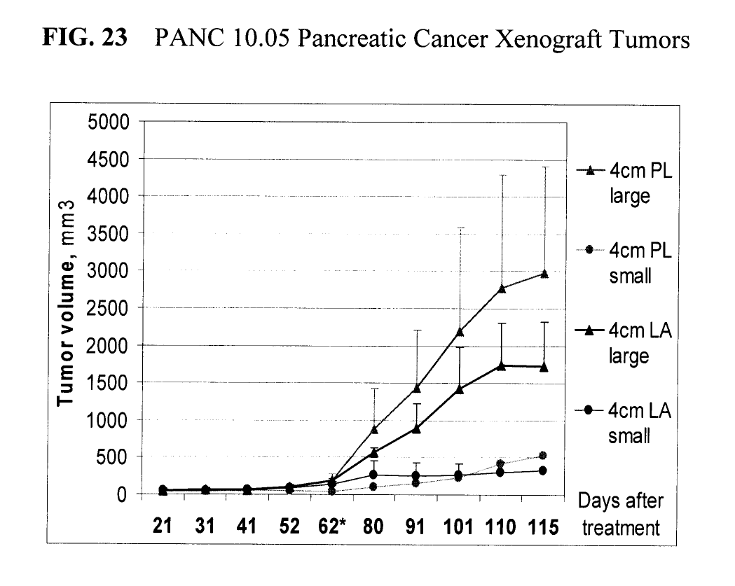

FIG. 23 presents results of an experiment in which about 3.0×10⁶ cells of the PANC 10.05 pancreatic cancer cell line were injected bilaterally into two groups (one treatment group and a control group), each with four mice. Seven days prior to the cell injection, a controlled-release leuprolide acetate formulation was implanted into each mouse in the treatment group. Four centimeters of leuprolide rod, providing 22.5 mg of leuprolide, were implanted in each mouse of the treatment group. Four centimeters of placebo rod (without leuprolide) were implanted one week prior to injection into each mouse of the control group.

FIG. 23 presents results of tumor xenograft growth over time in the placebo group and the leuprolide implant group. As FIG. 23 shows, tumor measurements were started on day 21 after injection. Tumor data was plotted according to the sizes of the tumors (large tumors: ≧500 mm³; small tumors: ≦500 mm³). By day 115 after injection, large tumors in the placebo group (n=4) had grown to approximately 3000 mm³ on average, while large tumors in the LA treatment group (n=3) had grown to 1700 mm³ on average. By day 115 after injection, the small tumor in the placebo group (n=1) had grown to approximately 500 mm³ on average, while small tumors in the LA treatment group (n=2) had grown to 400 mm³ on average.

### Experiment 24

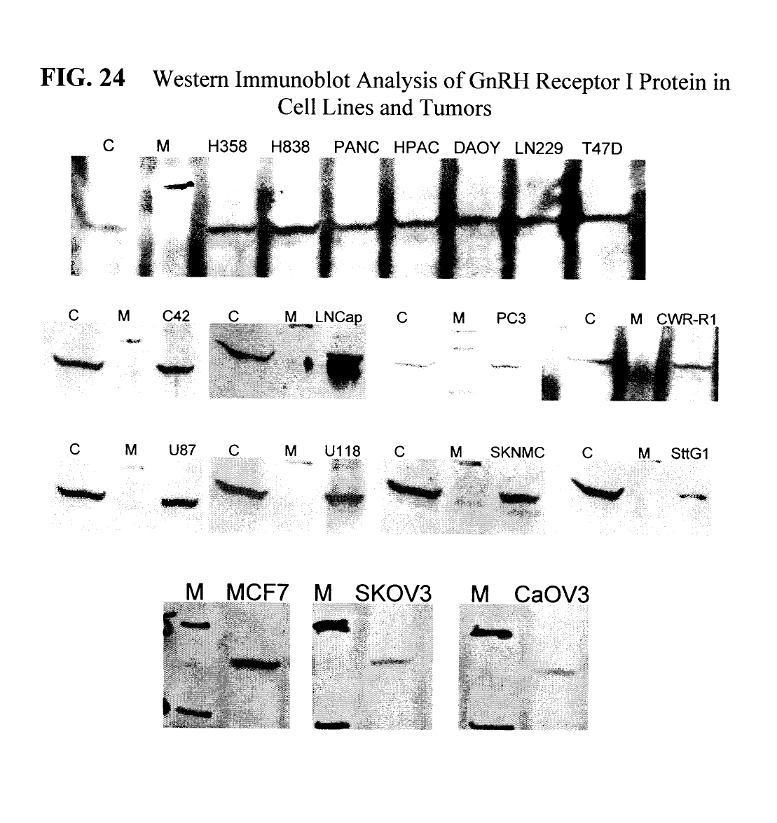

FIG. 24 presents results of protein expression studies to analyze the expression of the GnRH receptor I in various cancer cell lines and tumors. As described above, protein was extracted from cell lines and tumors and subjected to fractionation by denaturing polyacrylamide gel electrophoresis. Proteins were electroblotted to nitrocellulose membranes, and GnRH receptor I protein was detected by incubating membranes with rabbit antiserum directed against the human receptor, followed by incubation with a secondary antibody to rabbit. Chemiluminescence was used to visualize specific protein bands for GnRH receptor I. GnRH receptor I was detected in non-small cell lung carcinoma cell lines (H358, H838), pancreatic cancer cell lines (Panc, HPAC), brain cancer cell lines (DAOY, LN229, U118MG, U87MG, SKNMC, and SttG1), breast cancer cell lines (T47D, MCF-7), prostate cancer cell lines (LNCaP, C-42, PC3, and CWR-R1), and ovarian cancer cell lines (CaOV3 and SKOV3). “M” refers to molecular weight marker used for protein size determination and “C” refers to HPAC protein lysate used as a positive control of GnRH receptor I expression.

### Experiment 25

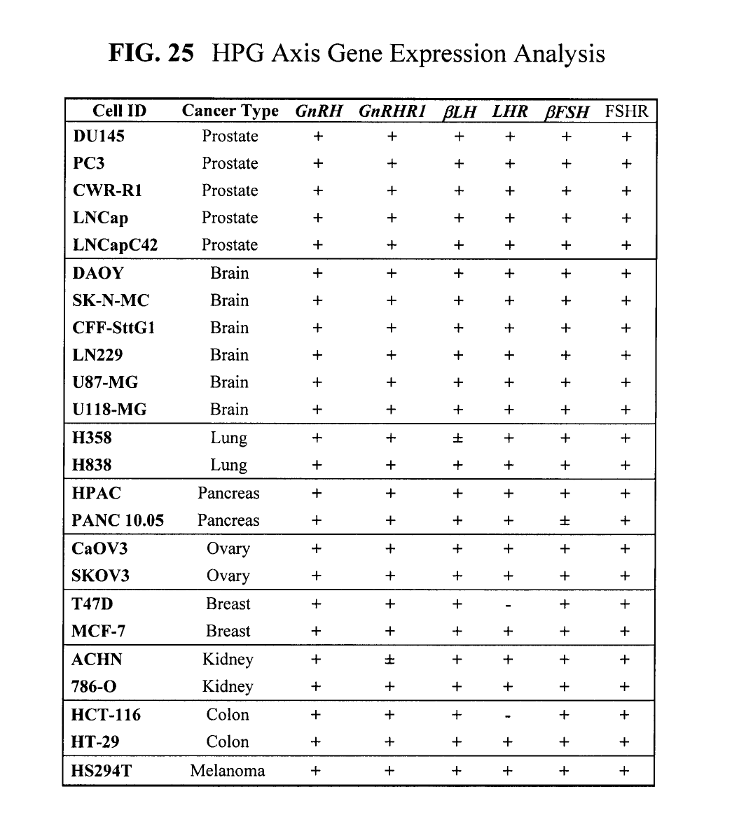

FIG. 25 presents results of gene expression studies to analyze the expression of GnRH, GnRH receptor I, LHβ, LH receptor, βFSH, and FSH receptor in various cancer cell lines. As described above, RNA was extracted from cell lines and tumors and subjected to enzymatic amplification of complementary DNAs. These complementary DNA samples were then amplified by PCR using specific primers for the genes listed above and a constitutively-expressed gene for glyceraldehyde-3-phosphate-dehydrogenase (GAPDH). Amplified DNAs were subjected to fractionation in 1.1% agarose (Tris-Borate-EDTA) gels and bands representing a fragment of the GnRH receptor I were visualized by staining with ethidium bromide. GnRH, GnRH receptor I, LHβ, LH receptor, βFSH, and FSH receptor were detected in prostate cancer cell lines (DU145, PC3, CWR-R1, LNCaP, and LNCaP-C42), brain cancer cell lines (DAOY, SKNMC, CFF-SttG1, LN229, U87MG, and U118MG), non-small cell lung carcinoma cell lines (H358, H838), pancreatic cancer cell lines (HPAC, Panc), ovarian cancer cell lines (SKOV3 and CaOV3), breast cancer cell lines (MCF-7, T47D), kidney cancer cell lines (ACHN, 786-O), colon cancer cell lines (HCT-116, HT-29), and a malignant melanoma cell line (HS294T). In FIG. 25, + indicates detectable bands of amplified DNA of the expected size based on the primer design; ± indicates DNA bands that were of less abundance compared to highly expressed genes.

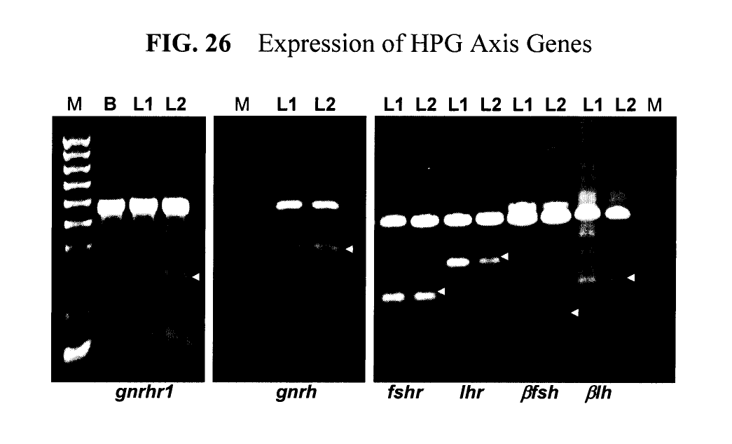

FIG. 26 demonstrates results of gene expression analysis in a breast cancer cell line (T47D), designated “B”, and non-small cell lung carcinoma cell lines (H358, H838), designated “L1” and “L2”, respectively, representative of the results achieved with reverse-transcriptase PCR. “M” refers to a DNA molecular weight marker. Arrowheads mark the PCR products of interest. Gnrhr1 refers to gonadotropin releasing hormone receptor-1, gnrh refers to gonadotropin releasing hormone, fshr refers to follicle stimulating hormone receptor, lhr refers to luteinizing hormone receptor, βfsh refers to follicle stimulating hormone, and βlh refers to luteinizing hormone.

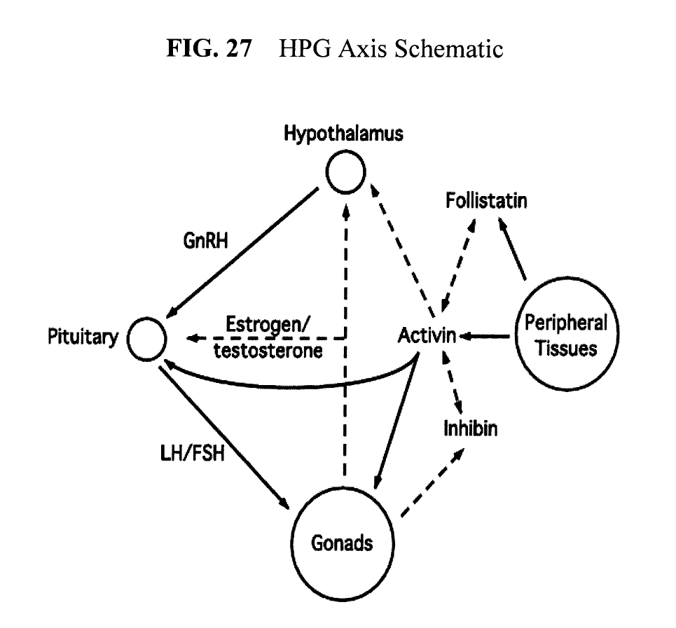

FIG. 27 is a schematic diagram of the HPG axis.

### Exemplary Embodiments

In embodiments of this invention, HPG axis-positive cancers are prevented, treated, delayed, or mitigated by administering high doses of at least one physiological agent that is a GnRH agonist or antagonist, effective to reduce local tissue production of hormones of the HPG axis or to down-regulate hormone receptors. In embodiments, the physiological agent is leuprolide, and the amount administered is sufficient to maintain the serum leuprolide levels at greater than about 1.5 ng/ml for the full dosing period. In other embodiments, the amount of leuprolide administered is sufficient to maintain the serum leuprolide levels at greater than about 2.0 ng/ml for the full dosing period. In other embodiments, the amount of leuprolide administered is sufficient to maintain the serum leuprolide levels at greater than about 2.5 ng/ml for the full dosing period. In other embodiments, the amount of leuprolide administered is sufficient to maintain the serum leuprolide levels at greater than about 3.0 ng/ml for the full dosing period.

In further embodiments, the physiological agent is an agent other than leuprolide, and the amount administered is an amount sufficient to maintain the serum levels of the agent at greater than about 1.5 ng/ml for the full dosing period, greater than about 2.0 ng/ml for the full dosing period, greater than about 2.5 ng/ml for the full dosing period, or greater than about 3.0 ng/ml for the full dosing period.

A “full dosing period” according to the present invention refers to a period of time sufficient to achieve a therapeutic effect in the treatment, mitigation, delay, or prevention of HPG axis-positive cancers, and may be from about one month to about twelve months, or such shorter or longer period of time as is required to achieve the therapeutic effect. In embodiments of the invention, the full dosing period is in the range of from about 30 days to about 90 days. In other embodiments, the full dosing period is about 60 days.

Since no toxic dose of GnRH agonists is believed to have been documented, other embodiments of this invention include treating, preventing, slowing the progression of, or mitigating HPG axis-positive cancers by continually increasing the dose of the GnRH agonist or antagonist until a decrease in a cancer-specific marker is achieved, or until the patient develops adverse effects that represent greater risk or discomfort than does the risk or discomfort of the cancer. Cancer-specific markers include or are expected to include, but are not limited to: dynein, α-PIX, and sorcin, which are proteins that have been shown to be differentially expressed in gliomas compared to normal brain; prostate-specific antigen (PSA); Ki67, a cell proliferation marker that decreases if cells slow in proliferation, and which is expected to be a useful marker for any cancer, including any HPG axis-positive cancer; and carcinoembryonic antigen (CEA), a marker for colon cancers.

In further embodiments of the invention, HPG axis-positive cancers would be prevented, treated, delayed, or mitigated by directly and constantly infusing GnRH agonists or antagonists into the affected tissue. It is well known in the art to deliver drugs by infusion through a catheter embedded directly in a part of a patient's body requiring treatment, for example, in the liver of a patient requiring chemotherapy drugs for the treatment of liver cancer.

In another embodiment of the invention, controlled release formulations of GnRH agonists or antagonists would be implanted directly into or near the cancer tissue in order to prevent, treat, delay, or mitigate HPG axis-positive cancers, for example by implantation directly into the tumor site following a surgical resection of a tumor. This would allow for high concentrations of the GnRH agonist or antagonist while minimizing peripheral exposure.

Currently, in the course of an in vitro fertilization process, a needle may be used to inject about 1 mg/day of GnRH agonists or antagonists into a patient. According to an embodiment of the present invention, a dose of a GnRH agonist or antagonist administered for the prevention, treatment, delay, or mitigation of HPG axis-positive cancers, when delivered by implantation of controlled release formulations directly into or near the tumor, results in serum levels of up to about 3 ng/ml or more, and is expected to result in tumor/organ tissue levels of up to about 3 ng/ml. In other embodiments of the present invention, the dosage regime of GnRH agonist or antagonist to treat, prevent, mitigate, or slow the progression of HPG axis-positive cancers would be a dose that is physiologically equivalent to a dose of leuprolide in the range of about 11.25 mg/month to about 22.5 mg/month, or a dose of an agent resulting in daily dosages physiologically equivalent to a dose of leuprolide of approximately 0.375 mg/day to approximately 0.75 mg/day. In additional embodiments, a controlled release formulation would be formulated to maintain a tissue concentration of the GnRH agonist or antagonist at levels that maintain the serum leuprolide levels at greater than about 1.5 ng/ml for the full dosing period. In other embodiments, the amount of leuprolide administered is sufficient to maintain the serum leuprolide levels at greater than about 2.0 ng/ml for the full dosing period. In other embodiments, the amount of leuprolide administered is sufficient to maintain the serum leuprolide levels at greater than about 2.5 ng/ml for the full dosing period. In other embodiments, the amount of leuprolide administered is sufficient to maintain the serum leuprolide levels at greater than about 3.0 ng/ml for the full dosing period. In embodiments of the invention, the higher tissue concentration would be substantially sustained at a high level instead of spiking initially and briefly to a very high level and then dropping substantially.

In other embodiments of the invention, an implanted controlled release formulation of GnRH agonists or antagonists would achieve a release profile that provides a substantially stable serum concentration of GnRH agonists or antagonists that is at least about two to about five times the serum concentration provided by currently-known cancer treatments using GnRH agonists or antagonists (for example, treatments for prostate cancer), with the serum concentration being substantially sustained at the higher level instead of spiking initially and briefly to a very high level and then dropping substantially as occurs with currently-known treatments. For example, an implanted controlled release formulation of the present invention for preventing, treating, delaying, or mitigating GnRH receptor-positive cancers would provide a GnRH agonist or antagonist serum concentration of at least about 1.5 ng/ml, in embodiments up to about 3.0 ng/ml or more over the lifetime of the formulation. Such formulations, using polymeric controlled release technology, are available from Durect Corporation, Cupertino, Calif. The lifetime of the implanted controlled release formulation according to the present invention may be from about one month to about twelve months, or such shorter or longer lifetime as is appropriate for the treatment, mitigation, delay, or prevention of HPG axis-positive cancers. In embodiments of the invention, the lifetime of the formulation is in the range of from about 30 days to about 90 days. In other embodiments, the lifetime of the formulation is about 60 days.

Other known methods of delivery are also suitable for administering GnRH agonists or antagonists according to the present invention, such as intramuscular injection of microspheres.

Examples of GnRH agonists or antagonists include but are not limited to Antide® brand of iturelix; Lupron® brand of leuprolide acetate; Zoladex® brand of goserelin acetate; Synarel® brand of nafarelin acetate; Treistar Depot brand of triptorelin; Supprelin brand of histrelin; Suprefact® brand of buserelin; Cetrotide® brand of cetrorelix; Plenaxis® brand of abarelix; Antagon brand of ganirelix; and degarelix (FE200486).

Embodiments of the present invention also include treating, mitigating, slowing the progression of, or preventing HPG axis-positive cancers by co-administering a GnRH agonist or antagonist with conventional chemotherapeutic treatment, the GnRH agonist or antagonist being administered in accordance with the treatment protocols described herein, or with modifications to the protocols that would be apparent to one of ordinary skill in the art in light of the present specification.

Embodiments of the present invention further include treating, mitigating, slowing the progression of, or preventing HPG axis-positive cancers by co-administering a GnRH agonist or antagonist with conventional radiation therapy, the GnRH agonist or antagonist being administered in accordance with the treatment protocols described herein, or with modifications to the protocols that would be apparent to one of ordinary skill in the art in light of the specification.

Embodiments of the present invention also include treating, mitigating, slowing the progression of, or preventing HPG axis-positive cancers by administering a GnRH agonist or antagonist prior to surgical resection of a tumor, the GnRH agonist or antagonist being administered in accordance with the treatment protocols described herein, or with modifications to the protocols that would be apparent to one of ordinary skill in the art in light of the present specification.

Embodiments of the present invention additionally include treating, mitigating, slowing the progression of, or preventing HPG axis-positive cancers by administering a GnRH agonist or antagonist during the immediate period after a surgical resection and indefinitely thereafter to prevent tumor recurrence, the GnRH agonist or antagonist being administered in accordance with the treatment protocols described herein, or with modifications to the protocols that would be apparent to one of ordinary skill in the art in light of the present specification.

Embodiments of the present invention also include treating, mitigating, slowing the progression of, or preventing HPG axis-positive cancers by co-administering a GnRH agonist or antagonist with LH receptor blockers or analogues thereof, which include but are not limited to interleukin-1 and anti-LH receptor immunoglobulins; co-administering a GnRH agonist or antagonist with activin receptor blockers or analogues thereof; and administering other agents, including agents not yet known, that decrease the degradation of, increase the half-life of, or increase tumor tissue levels of GnRH agonists or antagonists. In these embodiments, the GnRH agonist or antagonist would be administered in accordance with the treatment protocols described herein, or with modifications to the protocols that would be apparent to one of ordinary skill in the art in light of the present specification.

Additionally, the present invention encompasses pharmaceutical formulations containing GnRH agonists and/or GnRH antagonists and which are configured to be implanted in or near tumor tissue and to provide serum concentrations or certain tissue concentrations of the GnRH agonists and/or GnRH antagonists that are up to about 10 times higher than serum levels resulting from conventional cancer treatments using GnRH agonists or antagonists, such as, for example, conventional prostate cancer treatments. The pharmaceutical formulations could be used, for example, to treat, delay, mitigate, or prevent HPG axis-positive cancers.

While various embodiments of the present invention have been described throughout this specification, it should be understood that they have been presented by way of example only, and not by way of limitation. For example, the present invention is not limited to the agents illustrated or described. As such, the breadth and scope of the present invention should not be limited to any of the above-described exemplary embodiments, but should be defined in accordance with the appended claims and their equivalents.

What is claimed is:

1. A method for treating an HPG axis-positive cancer in a patient having an HPG axis-positive cancer, for preventing an HPG axis-positive cancer in a patient at risk of contracting an HPG axis-positive cancer, for decreasing the level of an HPG axis-positive cancer-specific marker in a patient, or for preventing or slowing proliferation of cells of HPG axis-positive cancer origin in a patient, comprising:
administering to the patient a therapeutically effective amount of at least one physiological agent that decreases or regulates blood or tissue levels, expression, production, function, or activity of at least one of luteinizing hormone (LH), LH receptors, follicle stimulating hormone (FSH), FSH receptors, an androgenic steroid, androgenic steroid receptors, an activin, and activin receptors.

2. A method for treating an HPG axis-positive cancer in a patient having an HPG axis-positive cancer, for preventing an HPG axis-positive cancer in a patient at risk of contracting an HPG axis-positive cancer, for decreasing the level of an HPG axis-positive cancer-specific marker in a patient, or for preventing or slowing proliferation of cells of HPG axis-positive cancer origin in a patient, comprising:
administering to the patient a therapeutically effective amount of at least one physiological agent that increases or regulates blood or tissue levels, expression, production, function, or activity of at least one of gonadotropin releasing hormone (GnRH), an inhibin, and a follistatin.

3. A method of preventing or inhibiting an upregulation of the cell cycle in HPG axis-positive cancer-derived cells in a patient, comprising:
administering to the patient an amount of at least one physiological agent selected from the group consisting of GnRH agonists and GnRH antagonists, effective to reduce local tissue production of hormones of the hypothalamic-pituitary-gonadal (HPG) axis.

4. A method of treating an HPG axis-positive cancer in a patient having an HPG axis-positive cancer, comprising:
administering to the patient an amount of at least one physiological agent selected from the group consisting of GnRH agonists and GnRH antagonists, effective to achieve a blood serum level of about 1.5 ng/ml of the physiological agent for a predetermined time interval.

5. A method of treating an HPG axis-positive cancer in a patient having an HPG axis-positive cancer, comprising:
administering to the patient an amount of at least one physiological agent selected from the group consisting of GnRH agonists and GnRH antagonists, effective to achieve a blood serum level of about 2.0 ng/ml of the physiological agent for a predetermined time interval.

6. A method of treating an HPG axis-positive cancer in a patient having an HPG axis-positive cancer, comprising:
administering to the patient an amount of at least one physiological agent selected from the group consisting of GnRH agonists and GnRH antagonists, effective to achieve a blood serum level of about 2.5 ng/ml of the physiological agent for a predetermined time interval.

7. A method of treating an HPG axis-positive cancer in a patient having an HPG axis-positive cancer, comprising:
administering to the patient an amount of at least one physiological agent selected from the group consisting of GnRH agonists and GnRH antagonists, effective to achieve a blood serum level of about 3.0 ng/ml of the physiological agent for a predetermined time interval.

8. A method for treating an HPG axis-positive cancer in a patient having an HPG axis-positive cancer, comprising:
administering to the patient an initial dose of a GnRH agonist or a GnRH antagonist; and
monitoring for decreases in an HPG axis-positive cancer-specific marker level in the patient, and subsequently administering to the patient increasing doses of the GnRH agonist or the GnRH antagonist until no further decrease in an HPG axis-positive cancer-specific marker level in the patient is observed.

9. A method for treating an HPG axis-positive cancer in a patient having an HPG axis-positive cancer, comprising:
administering to the patient a therapeutically effective amount at least one physiological agent selected from the group consisting of GnRH agonists and GnRH antagonists by substantially continuously infusing the physiological agent directly into an organ or anatomical site of the patient affected by the HPG axis-positive cancer so that HPG axis-positive cancer cells are exposed to concentrations of the physiological agent that would result from blood serum concentrations of the physiological agent of about 1.5 to about 3.0 ng/ml for a pre-determined time interval.

10. The method of claim 1, wherein the at least one physiological agent is one of gonadotropin releasing hormone (GnRH), a GnRH agonist, a GnRH antagonist, an inhibin, beta-glycan, and a follistatin.

11. The method of any one of claims 1-3, wherein the at least one physiological agent is leuprolide, and the therapeutically effective amount is in the range of about 11.25 mg/month to at least about 22.5 mg/month.

12. The method of any one of claims 1-3, wherein the therapeutically effective amount of the at least one physiological agent is an amount of the physiological agent, administered or released over a predetermined time period, targeted to achieve substantially equivalent physiological effects as those resulting from a blood serum level of leuprolide of between about 1.5 and about 3 ng/ml of leuprolide over a period of about two months.

13. A method for treating an HPG axis-positive cancer in a patient having an HPG axis-positive cancer, comprising:
administering to the patient a therapeutically effective amount of at least one physiological agent selected from the group consisting of GnRH agonists and GnRH antagonists, by implanting a pharmaceutical controlled release formulation of the at least one physiological agent directly into or near tissue of the patient affected by the HPG axis-positive cancer.

14. A method for treating an HPG axis-positive cancer in a patient having an HPG axis-positive cancer, comprising:
administering to the patient a therapeutically effective amount of at least one physiological agent selected from the group consisting of GnRH agonists and GnRH antagonists, by infusing a pharmaceutical controlled release formulation of the at least one physiological agent directly into tissue of the patient affected by the HPG axis-positive cancer.

15. The method of claim 13, wherein the pharmaceutical controlled release formulation is formulated to provide a serum concentration of the at least one physiological agent of between about 1.5 and about 3 ng/ml maintained for a period of at least about two months.

16. The method of claim 13, wherein the pharmaceutical controlled release formulation is formulated to expose HPG axis-positive cancer cells of the patient to concentrations of the at least one physiological agent resulting from a blood serum concentration of the at least one physiological agent of between about 1.5 and about 3 ng/ml for a period of at least about two months.

17. A method for treating an HPG axis-positive cancer in a patient having an HPG axis-positive cancer, comprising:
administering to the patient a first physiological agent selected from the group consisting of GnRH agonists and GnRH antagonists in a therapeutically effective combination with a second physiological agent selected from the group consisting of androgen synthesis blockers, analogues of androgen synthesis blockers, FSH receptor blockers, analogues of FSH receptor blockers, testosterone, testosterone analogues, LH receptor blockers, analogues of LH receptor blockers, activin blockers, and analogues of activin blockers.

18. A method for treating an HPG axis-positive cancer in a patient having an HPG axis-positive cancer, comprising:
administering to the patient having the HPG axis-positive cancer a physiological agent that decreases the degradation of GnRH agonists or GnRH antagonists within the patient, increases the half-life of GnRH agonists or GnRH antagonists within the patient, or increases tissue levels of GnRH agonists or GnRH antagonists within the patient.

19. A method for treating an HPG axis-positive cancer in a patient having an HPG axis-positive cancer, comprising:
administering to the patient having the HPG axis-positive cancer an amount of at least one physiological agent selected from the group consisting of GnRH agonists and GnRH antagonists, effective to achieve a blood serum level of between about 1.5 and about 3.0 ng/ml of the physiological agent for a predetermined time interval in combination with administering to the patient a standard chemotherapeutic agent as indicated for the HPG axis-positive cancer.

20. A method for treating an HPG axis-positive cancer in a patient having an HPG axis-positive cancer, comprising:
administering to the patient having the HPG axis-positive cancer an amount of at least one physiological agent selected from the group consisting of GnRH agonists and GnRH antagonists, effective to achieve a blood serum level of between about 1.5 and about 3.0 ng/ml of the physiological agent for a predetermined time interval in combination with administering to the patient a standard radiation treatment regimen as indicated for the HPG axis-positive cancer.


SEQUENCE LISTING (18 sequences)

SEQ ID NO: 1 - DNA, 20 bp, Artificial
  Artificial primer sequence
gaccttgtctggaaagatcc

SEQ ID NO: 2 - DNA, 20 bp, Artificial
  Artificial primer sequence
caggctgatcaccaccatca

SEQ ID NO: 3 - DNA, 20 bp, Artificial
  Artificial primer sequence
gggggagccaaaagggtcat

SEQ ID NO: 4 - DNA, 20 bp, Artificial
  Artificial primer sequence
gccccagcgtcaaaggtgga

SEQ ID NO: 5 - DNA, 24 bp, Artificial
  Artificial primer sequence
ccttattctactgacttcgtgcgt

SEQ ID NO: 6 - DNA, 25 bp, Artificial
  Artificial primer sequence
ggaatatgtgcaacttggtgtaagg

SEQ ID NO: 7 - DNA, 21 bp, Artificial
  Artificial primer sequence
ctgctgctgttgctgctgctg

SEQ ID NO: 8 - DNA, 21 bp, Artificial
  Artificial primer sequence
ggtggtcacaggtcaaggggt

SEQ ID NO: 9 - DNA, 22 bp, Artificial
  Artificial primer sequence
acggccggtctcactcgactat

SEQ ID NO: 10 - DNA, 20 bp, Artificial
  Artificial primer sequence
cgtggcctccaggagattga

SEQ ID NO: 11 - DNA, 21 bp, Artificial
  Artificial primer sequence
gattaagacatgccattctga

SEQ ID NO: 12 - DNA, 22 bp, Artificial
  Artificial primer sequence
tttattggtagccattaattct

SEQ ID NO: 13 - DNA, 25 bp, Artificial
  Artificial primer sequence
gacactccagtttttcttccttttc

SEQ ID NO: 14 - DNA, 20 bp, Artificial
  Artificial primer sequence
aggaatctgcatggtgagca

SEQ ID NO: 15 - DNA, 20 bp, Artificial
  Artificial primer sequence
gacctcccgaggaatgccat

SEQ ID NO: 16 - DNA, 20 bp, Artificial
  Artificial primer sequence
ggtgaggctggcttccatga

SEQ ID NO: 17 - DNA, 23 bp, Artificial
  Artificial primer sequence
gacagaaacttcatccactgtcc

SEQ ID NO: 18 - DNA, 22 bp, Artificial
  Artificial primer sequence
gccaggaatattaaattagatg